(19) United States
(12) Patent Application Publication
(10) Pub. No.: US 2003/0229134 A1
(43) Pub. Date: Dec. 11, 2003

(54) Methods for stimulating nervous system regeneration and repair by inhibiting phosphodiesterase type 4

(76) Inventors: Marie T. Filbin, New York, NY (US); Elena Nikulina, Astoria, NY (US)

Correspondence Address: FISH & NEAVE, 1251 AVENUE OF THE AMERICAS, 50TH FLOOR, NEW YORK, NY, 10020-1105, US

(21) Appl. No.: 10/414,506

(22) Filed: Apr. 14, 2003

Related U.S. Application Data
(63) Continuation of application No. PCT/US01/46846, filed on Nov. 2, 2001.
(60) Provisional application No. 60/245,319, filed on Nov. 2, 2000.

(51) Int. Cl. A61K 31/4015

(52) U.S. Cl. 514/424

(57) ABSTRACT

The invention relates to the novel identification of inhibitors of phosphodiesterase type 4 (“PDE4”) as agents which can reverse inhibition of neural regeneration in the mammalian central and peripheral nervous system. The invention provides compositions and methods using agents that can reverse the inhibitory effects on neural regeneration by regulating PDE4 expression. A composition comprising at least one PDE4 inhibitor in an amount effective to inhibit PDE4 activity in a neuron when administered to an animal is provided. Methods for regulating (e.g., promoting) neural growth or regeneration in the nervous system, methods for treating injuries or damage to nervous tissue or neurons, and methods for treating neural degeneration associated with disorders or diseases, comprising the step of administering to an animal a composition comprising a therapeutically effective amount of an agent which inhibits phosphodiesterase IV activity in a neuron are provided.

This application claims benefit of U.S. Provisional Application No. 60/245,319, filed Nov. 2, 2000, which is herein incorporated by reference.

### TECHNICAL FIELD OF THE INVENTION

The invention relates to the novel identification of inhibitors of phosphodiesterase type 4 (“PDE4”) as agents which can reverse inhibition of neural regeneration in the mammalian central and peripheral nervous system. The invention provides compositions and methods using agents that can reverse the inhibitory effects on neural regeneration by regulating PDE4 expression. A composition comprising at least one PDE4 inhibitor in an amount effective to inhibit PDE4 activity in a neuron when administered to an animal is provided. Methods for regulating (e.g., promoting) neural growth or regeneration in the nervous system, methods for treating injuries or damage to nervous tissue or neurons, and methods for treating neural degeneration associated with disorders or diseases, comprising the step of administering to an animal a composition comprising a therapeutically effective amount of an agent which inhibits phosphodiesterase IV activity in a neuron are provided.

### BACKGROUND OF THE INVENTION

Axons of the adult mammalian central nervous system (CNS) do not regenerate after injury despite the fact that there are many molecules present which encourage/promote axonal (nerve) growth. There are at least three factors that impede axonal regeneration: (1) the presence of specific inhibitors of axonal growth in myelin; (2) formation of a glial scar; and (3) the intrinsic growth state of the neurons. The glial scar takes some time after injury to form. Therefore, it would be advantageous to encourage growth in this “window-of-opportunity”, before the glial scar forms. The main obstacles immediately after injury, therefore, in the CNS as well as in the peripheral nervous system (PNS), are inhibitors of neuronal growth and regeneration present in myelin.

Despite the inability of axons of the adult mammalian CNS to regenerate after injury, when CNS neurons are placed in culture or when a permissive substrate is provided by grafting in peripheral nerve (Richardson et al., 1980, David and Aguayo, 1981) or embryonic spinal cord (Howland et al., 1995), those neurons can extend axons into, but not beyond, the permissive substrate. This suggests that the injured adult CNS environment inhibits axonal regeneration. Inhibitory molecules of the adult injured CNS identified to date are myelin-associated glycoprotein (MAG) (DeBellard et al., 1996; McKerracher et al., 1994; Mukhopadhyay et al., 1994) and Nogo (Chen et al., 2000; Spillmann et al., 1998). Other obstacles to axonal regeneration are proteoglycans secreted by reactive astrocytes and formation of a glial scar (McKeon et al., 1995).

Recent strategies for overcoming the neuronal growth inhibitors have included neutralizing the inhibitor or changing the growth capacity of the axons such that the axons no longer respond to myelin by being inhibited. In this way, they would resemble young axons which regenerate in vivo and which are not inhibited by myelin in vitro (see, e.g., U.S. Pat. Nos. 5,932,542 and 6,203,792, the entire disclosures of which are incorporated herein by reference).

Previous studies have demonstrated that neurons of the adult CNS can change their intrinsic ability to grow, i.e., they can be brought to a state where they do not respond to the inhibitors associated with myelin and can thus regenerate after injury (see, e.g., Bregman, 1998; Neumann and Woolf, 1999). Bregman and coworkers grafted a piece of embryonic spinal cord into the injured adult spinal cord and pumped neurotrophins into the graft. Significant axonal growth beyond the lesion was detected. Neumann and Woolf reported regeneration of central axons of dorsal root ganglion (DRG) neurons after a conditioning lesion to their peripheral branch. Other studies have demonstrated that elevating the endogenous levels of cyclic AMP (cAMP) in older neurons, either artificially with dibutyryl cAMP (dbcAMP) or by pre-treating the neurons with neurotrophins (“priming”), results in their not being inhibited by either myelin in general or by a specific myelin inhibitor, MAG. Cai et al., (1999); incorporated herein by reference. In addition, it has been shown that the endogenous level of cAMP in young neurons is very high and that their ability to regenerate in vivo and to grow on MAG and myelin is cAMP-dependent (Cai et al., 2001; see also U.S. Provisional Application No. 60/202,307, filed May 5, 2000 and PCT/US01/14364, filed May 4, 2001, claiming priority therefrom, the entire disclosures of which are incorporated herein by reference). More recently, it has been demonstrated that contacting a neural cell subject to growth repulsion mediated by a neural growth repulsion factor (e.g., myelin or MAG) with an activator of cyclic nucleotide dependent protein kinase promotes neural cell growth (see U.S. Pat. No. 6,268,352).

Another factor—the intrinsic ability of neurons to respond to the presence of these inhibitors—has also been the focus of several research groups. It is well-established that the embryonic CNS will regenerate (Hasan et al., 1993). Embryonic neurons are not inhibited by myelin in culture (Shewan et al., 1995) and can extend long axons when transplanted into the adult CNS (Li and Raisman, 1993). Additionally, it has been demonstrated that the levels of cAMP in neonatal DRG neurons are high and decrease dramatically at about postnatal day 3 (Cai et al., 2001). At about the same age, the rat spinal cord loses the ability to regenerate (Bregman, 1987; Bates and Stelzner, 1993). Therefore, there are currently few effective therapeutic agents or methods of promoting neural regeneration in injured or damages neurons.

It would be useful to be able to regulate the inhibitors of axonal regeneration in neurons for treating patients with nervous system conditions, injuries or degenerative disorders or diseases where neural regeneration is a problem. In particular, it would be useful to be able to induce or otherwise increase selectively cAMP activity in the mammalian nervous system—alone or in combination with other treatments—to relieve inhibition of axonal outgrowth by myelin and myelin inhibitors, such as myelin-associated glycoprotein (MAG).

### SUMMARY OF THE INVENTION

We have now shown that prolonged administration of a specific phosphodiesterase type 4 (“PDE4”) inhibitor reverses the normal inhibition of neural growth and regeneration in the central nervous system (CNS) and peripheral nervous system (PNS) mediated by myelin and myelin associated inhibitors such as MAG. Therefore, in one aspect, the invention provides pharmaceutical compositions comprising a PDE4 specific inhibitor in an amount effective to inhibit PDE4 activity in a neuron when administered to an animal, thereby relieving myelin- or MAG-mediated growth inhibition. In another aspect, the invention provides methods of administering a PDE4 specific inhibitor to a patient in order to reverse or prevent the normal inhibition of neural growth and regeneration in the CNS and PNS. Thus, the invention provides methods for regulating and for promoting (or repressing) neural growth or regeneration in the nervous system, methods for treating injuries or damage to nervous tissue or neurons, and methods for treating neural degeneration associated with injuries, conditions, disorders or diseases, such as diseases and injuries of the brain and spinal cord. Relief of MAG and myelin-mediated inhibition of neuronal growth and regeneration by using the methods of the present invention may also be used for therapeutic effect in a variety of neurodegenerative diseases and in disorders or conditions associated with memory loss. The invention also provides methods of prolonged administration of a PDE4 specific inhibitor to promote neuronal survival and to prevent glial scar formation. The invention also provides compositions and methods that regulate the inhibitory effects of myelin, and associated inhibitors such as MAG, on neural growth and regeneration by regulating (increasing or decreasing) PDE4 expression.

In one embodiment of the invention, the PDE4 specific inhibitor is one that crosses the blood-brain barrier, because it can be administered at a site that is distal from the site of neural injury or disease. In a preferred embodiment, the PDE4 inhibitor is rolipram, a small molecule that crosses the blood-brain barrier. In a more preferred embodiment, rolipram is administered subcutaneously. In another aspect, the invention provides methods for genetically decreasing PDE4 activity in order to reverse or prevent neural growth inhibition and regeneration, prevent glial scar formation and promote neuronal survival.

### BRIEF DESCRIPTION OF THE DRAWINGS

FIG. 1 shows that rolipram treatment in vitro partially blocks inhibition of neurite outgrowth by MAG. See Example 1. The black bars represent neurite outgrowth on MAG-expressing Chinese hamster ovary (CHO) cells and the striped bars represent outgrowth on a monolayer of control CHO cells (which do not express MAG). Lane 1: control (no addition of dbcAMP or rolipram); lane 2: 1 mM dbcAMP; lane 3: 0.1 uM rolipram; lane 4: 0.25 uM rolipram; lane 5: 0.5 uM rolipram; and lane 6: 1.0 uM rolipram.

FIGS. 2A and 2B show that priming with rolipram in vitro overcomes inhibition of axonal growth by MAG (FIG. 2A) or myelin (FIG. 2B). See Example 2. In FIG. 2A, the black bars represent neurite outgrowth on MAG-expressing Chinese hamster ovary (CHO) cells and the striped bars represent outgrowth on a monolayer of control CHO cells. FIG. 2A: lane 1: control; lane 2: 200 ng/ml BDNF; lane 3: 0.1 uM rolipram; lane 4: 0.25 uM rolipram. FIG. 2B: lane 1: control; lane 2: 200 ng/ml BDNF; lane 3: 0.25 uM rolipram.

FIGS. 3A and 3B show that subcutaneous injection of postnatal day 12 (P12) rats with rolipram overcomes inhibition of axonal outgrowth by MAG in vitro for cerebellar neurons (FIG. 3A) and dorsal root ganglia (FIG. 3B). See Example 3A. The black bars represent neurite outgrowth on MAG-expressing Chinese hamster ovary (CHO) cells and the striped bars represent outgrowth on a monolayer of control CHO cells. FIG. 3A: lane 1: control; lane 2: 1 mM dbcAMP; lane 3: 7.5 nmol/kg rolipram; lane 4: 25 nmol/kg rolipram; lane 5: 40 nmol/kg rolipram; lane 6: 75 nmol/kg rolipram. FIG. 3B: lane 1: control; lane 2: 1 mM dbcAMP; lane 3: 40 nmol/kg rolipram; lane 4: 75 nmol/kg rolipram.

FIG. 4 shows that subcutaneous injection of postnatal day 30 (P30) rats with rolipram overcomes inhibition of axonal outgrowth by MAG in vitro. See Example 3A. The black bars represent neurite outgrowth on MAG-expressing Chinese hamster ovary (CHO) cells and the striped bars represent outgrowth on a monolayer of control CHO cells. Lane 1: control; lane 2: 0.1 umol/kg rolipram; lane 3: 0.2 umol/kg rolipram; lane 4: 0.5 umol/kg rolipram; lane 5: 1.0 umol/kg rolipram; lane 6: 2.0 umol/kg rolipram.

FIG. 5 shows that repeated subcutaneous injection of P30 rats with rolipram blocks inhibition of neurite outgrowth by MAG. See Example 3A. The black bars represent DRG neuron neurite outgrowth on MAG-expressing Chinese hamster ovary (CHO) cells and the striped bars represent outgrowth on a monolayer of control CHO cells. Lane 1: control; lane 2: three injections of rolipram every three hours, neurons isolated 20 hours after the last injection; lane 3: two injections of rolipram every three hours, neurons isolated 3 hours after the last injection; lane 4: injections every three hours for one day; neurons isolated at day 1; lane 5: injections every three hours for two days; neurons isolated at end of second day; lane 6: injections every three hours for three days; neurons isolated at end of third day.

FIG. 6 shows that rolipram delivered subcutaneously by minipump blocks inhibition of neuronal outgrowth by MAG in vitro. See Example 3B. The black bars represent neurite outgrowth on MAG-expressing Chinese hamster ovary (CHO) cells and the striped bars represent outgrowth on a monolayer of control CHO cells. Lane 1: control; lane 2: 0.4 umol/kg/hour rolipram; lane 3: 0.5 umol/kg/hour rolipram; lane 4: 0.7 umol/kg/hour rolipram.

FIG. 7 shows that rolipram delivered subcutaneously by minipump progressively blocks inhibition of neuronal outgrowth by MAG in vitro over time. See Example 3B. The black bars represent neurite outgrowth on MAG-expressing Chinese hamster ovary (CHO) cells and the striped bars represent outgrowth on a monolayer of control CHO cells. Lane 1: control; lane 2: 1 day of rolipram treatment; lane 3: 2 days of rolipram treatment; lane 4: 3 days of rolipram treatment.

FIG. 8 shows that rolipram delivered subcutaneously by minipump promotes motor neuron recovery in the presence of a Schwann cell bridge in vivo in rats after complete spinal cord transection. See Example 4A. Squares: 0.07 umol/kg/hour rolipram; diamonds: 10 mM dbcAMP; triangles: saline control.

FIG. 9 shows that rolipram delivered subcutaneously by minipump promotes motor recovery in vivo in rats after a moderate spinal cord contusion. See Example 4B. Xs: 0.07 umol/kg/hour rolipram plus Schwann cell transplantation and 4 injections, each of 0.2 ul, of 1 mM dbcAMP one week after injury; squares: 4 injections, each of 0.2 ul, of 1 mM dbcAMP one week after injury; triangles: 4 injections, each of 0.2 ul, of 50 mM dbcAMP one week after injury; circles: 4 injections, each of 0.2 ul, of 1 mM dbcAMP one day after injury; diamonds: Schwann cell transplantation one week after injury.

### DETAILED DESCRIPTION OF THE INVENTION

In order that the invention herein described may be fully understood, the following detailed description is set forth.

Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, neurobiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press (1989); Sambrook et al., Molecular Cloning: A Laboratory Manual, 3d ed., Cold Spring Harbor Press (2001); Ausubel et al., Current Protocols in Molecular Biology, Greene Publishing Associates (1992, and Supplements to 2001); Ausubel et al., Short Protocols in Molecular Biology: A Compendium of Methods from Current Protocols in Molecular Biology, 4th Ed., Wiley & Sons (1999); Harlow and Lane, Antibodies: A Laboratory Manual, Cold Spring Harbor Laboratory Press (1990); Harlow and Lane, Using Antibodies: A Laboratory Manual, Cold Spring Harbor Laboratory Press (1999); Crawley et al., Current Protocols in Neuroscience, John Wiley and Sons (1997 and supplements to 2001); and Kleitman et al., Culturing Nerve Cells, pp. 337-78, MIT Press, Cambridge, Mass./London, England (G. Banker and K. Goslin, Eds.) (1991); each of which is incorporated herein by reference in its entirety.

Enzymatic reactions and cell culture and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

The term “PDE4” refers to a brain-enriched isoform of phosphodiesterase, an enzyme that catalyzes the hydrolytic conversion of cAMP to AMP. Such conversion may be assessed by any of a number of methods well known to those of skill in the art, including enzymatic assays using a labeled or otherwise detectable substrate. The present invention provides methods and compositions comprising inhibitors of PDE4 on amounts that are effective in relieving myelin- or MAG-mediated inhibition of neuronal growth in the mammalian CNS or PNS. Preferably, a PDE4 inhibitor according to the invention is administered subcutaneously to a mammalian subject.

The term “PDE4 inhibitor” (also “PDE inhibitory activity”) refers to an inhibitor that measurably reduces the activity of a PDE4 enzyme. The term “PDE4 specific inhibitor” refers to an inhibitor that reduces the activity of a PDE4 enzyme preferentially to that of another enzyme, particularly that of another PDE enzyme. In a preferred embodiment, a PDE4 specific inhibitor is one that inhibits PDE4 activity at least 5-fold greater than it inhibits another PDE enzyme. In a more preferred embodiment, a PDE4 specific inhibitor is one that inhibits PDE4 activity at least 1 O-fold greater, more preferably at least 20-fold greater, even more preferably at least 50-fold greater than the inhibitor inhibits another PDE enzyme. PDE enzymatic assays are well known to those of skill in the art. Preferably, a PDE4 inhibitor of the invention affects one or more characteristics of PDE4 activity, e.g., association and dissociation constants, catalytic rates and substrate turnover rates, in a direction which reduces the overall PDE4 activity in a neuron compared to PDE4 in the absence of the putative inhibitor.

An agent which alters or modulates the PDE4 “activity”, “bioactivity” or “biological activity” in a neuron refers to an agent which can directly or ultimately increase (agonist) or decrease (antagonist) PDE4 enzymatic activity (the conversion of cAMP to AMP) in a neuron. PDE4 activity may be modulated by altering levels of PDE4 expression, i.e., by altering DNA, RNA or protein encoding PDE4 or a PDE4 modulatory agent in a neuron. PDE4 activity may also be modulated by mutation or alteration of a PDE4 polynucleotide or polypeptide molecule directly. Such mutations or alterations include, but are not limited to, those which alter a substrate affinity constant or binding rate, a substrate dissociation rate, the catalytic or turnover rate of the enzyme, and the binding constant of a PDE4 subunit to another homologous or heterologous subunit or molecule which affects (increases or decreases) catalysis by the PDE4 molecule. One having ordinary skill in the art would be readily able to determine whether a compound was a PDE inhibitor, a specifically a PDE4 inhibitor, using methods known in the art. See, e.g., Allen et al. (1999), herein incorporated by reference, which discloses a method for evaluating inhibitors of PDE4. See also, Kit Number TRKQ7090 from Amersham, which provides assays for PDE.

PDE4 activity in a neuron may also be modulated by association (covalent or non-covalent) with another agent or factor, e.g., an agonist or antagonist. The direction and magnitude of a putative PDE4 modulatory agent or modulator may be determined by measuring PDE4 activity in the absence and presence of the putative modulator, preferably in a time- and dose-dependent manner, using methods well known to the art.

PDE4 activity may be measured directly by PDE4 specific enzymatic assays (as described supra) or indirectly by assaying PDE4 encoding nucleic acid levels in a cell (e.g., by RT-PCR, Northern blot analysis or other methods for measuring levels of steady-state RNA encoding arginase), or PDE4-specific protein molecules in a cell (e.g., by a variety of immunoaffinity procedures, including Western blot techniques, ELISA assays and the like)—all of which are techniques that are well-known to those of skill in the art and which are described herein. Similarly, nucleic acid or protein molecules whose expression levels correlate with PDE4 activity in a cell may be used to measure PDE4 indirectly.

A PDE4 inhibitor is one that at an effective dose inhibits PDE4 activity by at least 10-fold compared to PDE4 activity in the absence of the inhibitor. In a preferred embodiment, a PDE4 inhibitor is one that at an effective dose inhibits PDE4 activity by at least 20-fold, more preferably 50-fold or even at least 100-fold.

The terms “axonal growth” or “axonal regeneration” as used herein refer both to the ability of an axon to extend in length and to the ability of an axon to sprout. An axon sprout is defined as a new process that extends from an existing or growing axon. (See, e.g., Ma et al., Nat. Neurosci., 2, pp. 24-30 (1999), which is incorporated herein by reference).

The term “MAG” refers to myelin-associated glycoprotein, which is a molecule derived from myelin which promotes or inhibits neuronal growth and regeneration in the CNS and PNS depending on the cell type and the developmental stage of the neuron. The term “MAG” also refers to a “MAG derivative”, which is a molecule comprising at least one MAG extracellular domain, wherein the MAG molecule has been altered (e.g., by recombinant DNA techniques to make chimera with portions of other molecules fused to the MAG molecule, or by chemical or enzymatic modification) or mutated (e.g., internal deletions, insertions, rearrangements and point mutations). MAG derivatives, unless otherwise noted, retain MAG activity.

The term “neurotrophin” refers to a trophic factor that helps a neuron survive or grow. A neurotrophin elevates cyclic AMP (cAMP) levels in a neuron.

The term “patient” includes human and veterinary subjects.

A “trophic factor” is a substance that helps a cell survive or grow and which elevates cyclic AMP (cAMP) levels.

A non-hydrolyzable cyclic AMP (cAMP) analog is a cAMP having a phosphodiesterase-resistant linkage and which therefore has greater bioactivity than an unmodified cAMP molecule. Examples include dibutyryl cAMP (dbcAMP) (Posternak and Weimann, Methods Enzymol., 38, pp. 399-409 (1974); incorporated herein by reference); and Sp-cAMP (Dostmann et al., J. Biol. Chem., 265, pp. 10484-491 (1990); incorporated herein by reference).

As used herein the phrase “therapeutically-effective amount” means an amount of a PDE4 modulatory agent of the invention such that the subject shows a detectable improvement in neuronal growth or regeneration after being treated under the selected administration regime (e.g., the selected dosage levels and times of treatment).

The term “treating” is defined as administering, to a subject, a therapeutically-effective amount of a compound of the invention, to prevent the occurrence of symptoms, to control or eliminate symptoms, or to palliate symptoms associated with a condition, disease or disorder associated with neuronal death or lack of neuronal growth.

The term “prolonged”, “prolonged administration”, or “prolonged treatment” as used herein, means administration of a compound, preferably a PDE4 specific inhibitor, for at least 12 hours, more preferably 24 hours, even more preferably 48, 72 or 96 hours. Prolonged treatment or administration may be for longer as well; including administration or treatment for up to one week, ten days, two weeks, one month, three months or six months.

The term “subject”, as described herein, is defined as a mammal or a cell in culture. In a preferred embodiment, a subject is a human or other animal patient in need of treatment.

A “BBB Score” is the result of a test developed by Basso, Beattie and Bresnahan as a modifed 21-point open field locomoter scale on which to measure the extent of recovery of motor function in rats with after spinal cord injury. See, e.g, Basso et al., J. Neurotrauma 13(7):343-59 (1996); Beattie et al. (1997), herein incorporated by reference.

Throughout this specification and claims, the word “comprise,” or variations such as “comprises” or “comprising,” will be understood to imply the inclusion of a stated integer or group of integers but not the exclusion of any other integer or group of integers.

Methods of Treating Nervous System Injury and Disease

The mammalian central nervous system does not regenerate after injury because, although there are many molecules present that promote and encourage a nerve to grow, there are also molecules present in the adult CNS that will actively prevent a nerve from regenerating. Thus, the result of nervous system injury can be paralysis or brain damage. Further, even though certain molecules have been identified as those which prevent neural regeneration, few treatments have been attempted in humans after spinal cord injury, largely because there is usually some partial function remaining, as a result of spared axons. Surgeons are thus reluctant to attempt any therapy that involves intervention at the injury site to avoid further damage, resulting in loss of what little function remains.

In animals, a number of treatments for nervous system injury have been somewhat successful, however, these are either not suitable or not ideal for use in humans (Bregman et al., 1995; Huang et al., 1999; Lehmann et al., 1999; McKerracher, 2001; Schnell and Schwab, 1990). For example, it has been shown that in mice immunized with myelin prior to when their spinal cords are lesioned, there is substantial regeneration and functional recovery (Huang et al., 1999). This treatment is not suitable for humans because, first, the treatment is necessary before the injury and when an injury is going to occur cannot be predicted. Second, immunization with myelin in humans is very likely to induce an autoimmune disease, multiple sclerosis, as it does in some strains of mice. Other treatments in animals require direct intervention at the injury site, which runs the risk of additional damage.

Applicants have addressed this problem by providing methods of using PDE4 inhibitors to treat nervous system injury and disease. Applicants have determined that inhibiting PDE4 in a neuron relieves inhibition of neuronal growth by myelin, and myelin inhibitors such as MAG. This invention is useful for treatment of nervous system injury—both of the peripheral nervous system (PNS) and central nervous system (CNS), particularly for CNS injury. Using the methods described herein, the inhibitory effects of myelin and MAG can be partially or fully blocked or relieved by agents that decrease or abolish PDE4 activity in a neuron. These agents, or modified forms of these or other agents which can modulate the activity of PDE4 in a neuron, may be administered to damaged nerves, directly or indirectly, alone or in combination, to reverse the inhibitory effects of myclin or myelin inhibitors such as MAG in vivo and to allow regeneration to proceed.

In one aspect, the invention provides a method of treating nervous system injury using PDE4 inhibitors. Nervous system injuries include, without limitation, spinal cord injury, brain injury, aneurysms, strokes and PNS injuries. In one embodiment, the invention provides a method of using an inhibitor that is specific for PDE4, which is expressed at high levels in the CNS. The advantage of using a PDE4 specific inhibitor is that it can be used to target the action of the inhibitor to the nervous system. Further, because PDE4 is not expressed at high levels in other tissues and organs of the mammal, treatment with PDE4 specific inhibitors will have fewer side effects than treatment with non-specific PDE inhibitors.

In a preferred embodiment, the method uses a PDE4 inhibitor that can be administered distal to the site of injury because an ideal treatment for treating patients with nervous system injury would be one that is the least invasive. In a more preferred embodiment, the method uses a PDE4 inhibitor that can be administered subcutaneously or intravenously, wherein the PDE4 inhibitor is one that is able to be effective at the site of injury. In the case of a brain or spinal cord injury, one highly preferred embodiment is a method that uses a PDE4 inhibitor that crosses the blood brain barrier and reaches the site of a CNS injury. In a preferred embodiment, the method uses the PDE4 inhibitor rolipram.

Applicants have found that prolonged treatment with a PDE4 inhibitor, particularly a PDE4 specific inhibitor, increases neuronal growth in a nerve cell. Not only does the PDE4 specific inhibitor relieves the inhibition of neuronal growth by myelin, MAG and other neuronal growth inhibitors, but prolonged treatment also promotes neuronal growth in the absence of neuronal growth inhibitors. Thus, in a preferred embodiment, the method comprises administering a PDE4 inhibitor for a prolonged period of time. In one embodiment, the method comprises administering a PDE4 inhibitor for at least 12 hours, more preferably at least 24, 48, 72 or 96 hours, even more preferably at least one week, two weeks, one month, two months or three months. The method comprises administering a PDE4 inhibitor for up to six months or 12 months. In a highly preferred embodiment of the invention, the method comprises administering a PDE4 inhibitor until the patient's nervous system injury is palliated or treated, or until the administration of the PDE4 inhibitor has no further beneficial effect. In a preferred embodiment, the PDE4 inhibitor is administered for three days to six months, one week to three months, or two weeks to one month.

Prolonged treatment may be accomplished by continuous administration of an effective amount of a PDE4 inhibitor sufficient to treat the nervous system disease or disorder, e.g., via a minipump, an implantable slow-release form of the inhibitor or intravenous drip administration. Alternatively, prolonged treatment may be accomplished by repeatedly administering an amount of the inhibitor at a dose level and dosage interval such that the PDE4 inhibitor concentration in the serum or cell or tissue of interest (e.g., a nervous system tissue or cell) never drops below the concentration that is required to treat the nervous system disease or disorder. Methods of determining the pharmacokinetic profiles of a particular compound are well-known in the art and may be used to determine the precise dose and dosage interval required to maintain the effective concentration. Repeated administration may be accomplished e.g., by administration once every 10 minutes, once every 30 minutes, once an hour, once every three hours, once every six hours or once every eight hours.

In another aspect, the invention provides methods for treating nervous system diseases by administering a PDE4 inhibitor to a patient in need thereof. In one embodiment, the methods of the invention are used for treating neural degeneration associated with disorders, conditions or diseases associated with apoptosis, necrosis or other forms of cell death. In a preferred embodiment, the methods are used to treat, without limitation, amyotrophic lateral sclerosis, Alzheimer's disease, Parkinson's disease, Huntington's disease, Creutzfeldt-Jacob disease, kuru, multiple system atrophy, amyotropic lateral sclerosis (Lou Gehrig's disease), and progressive supranuclear palsy. In another embodiment, the invention provides methods for treating a neural disease associated with viral infection (e.g., by herpes virus or HIV), encephalitis (viral or non-viral), mitochondrial disease, kuru and peripheral neuropathies.

Long-term potentiation, which is an animal model of memory acquisition, is cAMP-dependent, transcription-dependent and results in sprouting of axons (see, e.g., Ma et al., Nat. Neurosci., 2, pp. 24-30 (1999) (incorporated herein by reference)). Further, there are many molecular and morphological similarities between the cAMP-dependent ability of neurotrophins and dbcAMP to overcome inhibition by MAG and myelin and the changes associated with memory and learning (Bach et al., Proc. Natl. Acad. Sci. U.S.A., 96, pp. 5280-85 (1999); incorporated herein by reference). Thus, in another embodiment, the invention provides methods for treating memory and learning defects and disorders associated with neuronal death or lack of neuronal growth by administering a PDE4 inhibitor to a patient in need thereof.

In a preferred embodiment, the method of treating a nervous system disease uses a PDE4 inhibitor that does not have to be administered to the affected neural tissue. As described above, in a more preferred embodiment, the method uses a PDE4 inhibitor that can be administered subcutaneously or intravenously, wherein the PDE4 inhibitor is one that can reach the affected neural tissue. For both CNS and PNS diseases, one highly preferred embodiment is a method that uses a PDE4 inhibitor. It is especially preferred for CNS disease, that the method uses a PDE4 inhibitor that crosses the blood brain barrier. In a preferred embodiment, the method uses the PDE4 inhibitor rolipram.

In a preferred embodiment, the method for treating the nervous system disease or disorder comprises administering a PDE4 inhibitor for a prolonged period of time. In one embodiment, the method comprises administering a PDE4 inhibitor for at least one week, two weeks, one month, two months or three months. In a preferred embodiment, the method comprises administering a PDE4 inhibitor for six months or one year or more, especially in the case of chronic nervous system diseases. In a highly preferred embodiment of the invention, the method comprises administering a PDE4 inhibitor until the patient's nervous system disease or disorder is palliated, treated or stabilized, or until the administration of the PDE4 inhibitor has no further beneficial effect.

The methods of the invention may be used to treat injuries, diseases or disorders include traumatic spinal cord injury, traumatic brain injury, aneurysms and strokes. Such injuries, diseases or disorders also include PNS injury, viral infection (e.g., by herpes virus or HIV), encephalitis (viral or non-viral), mitochondrial disease, Creutzfeldt-Jacob disease, kuru, multiple system atrophy, peripheral neuropathies, diabetic neuropathy, periventricular leukomalacia associated with prematurity in infants, Guillian Barre syndrome, Pelizus Mersbecker, Dejerene-Sottas and progressive supranuclear palsy.

The methods of the invention may also be used to treat neurodegenerative diseases that include, but are not limited to: amyotropic lateral sclerosis (Lou Gehrig's disease; “ALS”); Parkinson's Disease; Parkinson's Plus Syndromes; ALS-Parkinson dementia complex; Huntington's Disease; Hodgkin's Disease; Alzheimer's Disease; Pick Disease; Wilson's Disease; hepatolenticular degeneration; environmental toxins, including manganese and carbon monoxide poisoning; inherited epilepsies; nutritional deficiency states (e.g., Wernicke-Korsakoff syndrome, B 12 deficiency and pellagra); prolonged hypoglycemia or hypoxia; paraneoplastic syndromes; heavy metal exposure (e.g., arsenic, bismuth, gold, manganese and mercury); dialysis dementia; Schilder disease; lipid-storage diseases; cerebrocerebellar degeneration; dementia with spastic paraplegia; progressive supranuclear palsy; Binswanger Disease; brain tumor or abcess; Marchiava-Bignami Disease, communicating, normal pressure or obstructive hydrocephalus; progressive multifocal leukoencephalitis; Lewy-Body Disease; some cases of AIDS; progressive aphasia syndromes; and frontal lobe dementia. See Principles of Neurology (Sixth Edition), Adams, R. D., Victor, M., and Ropper, A. H. eds. 1997 (McGraw-Hill, New York); incorporated herein by reference in its entirety.

Formation of the glial scar is another factor that contributes to the lack of regeneration in the CNS. The main components of the glial scar are reactive astrocytes and connective tissue elements that can serve as a scaffold for depositing various inhibitory molecules such as proteoglycans. Importantly, proliferation of astrocytes is blocked in response to elevated cAMP levels (see, e.g., Dugan et al, 1999), and the proliferation rate and extracellular matrix production capacity of invading fibroblasts is inhibited by elevated cAMP levels (Hermann et al. 2001). Thus, in another aspect, the invention provides methods of reducing or preventing glial scar formation after nervous system injury by administering a PDE4 inhibitor.

In a preferred embodiment, the method for preventing or reducing glial scar formation comprises administering a PDE4 inhibitor for a prolonged period of time. In one embodiment, the method comprises administering a PDE4 inhibitor for at least three days, one week, two weeks, one month, two months or three months after the injury has occurred. In a preferred embodiment, the method comprises administering a PDE4 inhibitor within a short period of time after the nervous system injury in order to prevent or reduce glial scar formation.

In another aspect, the properties of MAG as a negative axonal guidance cue can be used to guide regenerating axons to their correct target and keep them on the correct path. For this purpose, a PDE4 modulator of the invention, or modified forms of these or other agents that can alter (e.g., decrease or increase) PDE4 levels in a neuron are administered to the precise regions of the regenerating nervous tissue to encourage or contain growth along exact pathways.

PDE4 Inhibitors

The invention also provides a variety of inhibitors of PDE4 that may be used in the methods and compositions of the invention. A variety of inhibitors specific for PDE4 have been described. For a recent review, see V. Dal Piaz and M P Giovannoni, Eur. J. Med. Chem., May 2000;35(5): 463-80. See also, e.g., Dinter et al., J. Neuroimmunol., Aug. 1, 2000;108(1-2):136-46 (disclosing a selective PDE 4 inhibitor “mesopram”); Campos-Toimil et al., Arterioscler. Thromb. Vasc. Biol., September 2000;20(9):E34-40 (disclosing the effects of Gingko biloba extract EGb 761 as a PDE4 inhibitor); Ikamura et al., J. Pharmacol. Exp. Ther., August 2000;294(2):701-6 (disclosing rolipram or Ro-20-1724 as PDE4 specific inhibitors); Laliberte et al., Biochemistry, May 30, 2000;39(21):6449-58 (Rolipram); D. Haffner and P G Germann, Am. J. Respir. Crit. Care Med., May 2000;161(5):1495-500 (disclosing a the (PDE-4) inhibitor “roflumilast”); Banner et al., Clin. Exp. Allergy May 2000;30(5): 706-12 (disclosing PDE4 inhibitors CDP840, rolipram and RO-20-1724), Ehinger et al., Eur. J. Pharmacol., Mar. 24, 2000;392(1-2):93-9 (disclosing PDE4 inhibitor RPR 73401); Boichot et al., J. Pharmacol. Exp. Ther., February 2000;292(2):647-53 (disclosing adenine derivatives substituted in position 9 as selective PDE4 inhibitors); Souness et al., Biochem. Pharmacol., Sep. 15, 1999;58(6):991-9 (disclosing rolipram, RP 73401 (piclamilast), and other structurally diverse PDE4 inhibitors); He et al., (disclosing a series of 2,2-disubstituted indan-1,3-dione-based PDE4 inhibitors, and the RP 73401 and CDP 840 PDE4 inhibitors); and Dal Piaz et al., (disclosing a series of 6-aryl-4,5-heterocyclic-fused pyridazinones as selective phosphodiesterase PDE4 inhibitors); all of which are herein incorporated by reference. Preferred inhibitors of brain PDE4 include, but are not limited to, rolipram (Genain et al., Proc. Natl. Acad. Sci. U.S.A., Apr. 11, 1995;92(8):3601-5); Ro 20-1724 (Fujimaki et al., Neuropsychopharmacology, January 2000;22(1):42-51); and BBB022A (Falcik et al., J. Neuroimmunol., Jun. 1, 1999;97(1-2): 119-28). Also included are derivatives and analogs of the foregoing that inhibit PDE4.

In another aspect, one having ordinary skill in the art may use any compound that has PDE4 inhibitory activity in the methods and compositions of the invention. One may use any method to determine whether a compound has PDE4 inhibitory activity. Such methods are described supra. Further, one may determine whether a compound is a PDE4 specific inhibitor as described above.

Pharmaceutical Compositions of Neuronal PDE4 Modulators

The PDE4 modulatory agents of this invention may be formulated into pharmaceutical compositions and administered in vivo at an effective dose to treat the particular clinical condition addressed. In a preferred embodiment, the PDE4 modulatory agent is a PDE4 inhibitor, preferably a PDE4 specific inhibitor. In a more preferred embodiment, the pharmaceutical composition is one that is suitable for intravenous or subcutaneous administration, preferably one that is suitable for subcutaneous administration. In an even more preferred embodiment, the composition is one that is suitable for prolonged administration. In another preferred embodiment, the composition is contained within a device that permits prolonged administration. Such devices include, inter alia, minipump, slow-release oral or buccal tablets, transdermal patches, intravenous drip bags, rectal or vaginal suppositories, implantable slow-release gels, tablets or erodable biomatrices. Administration of one or more of the pharmaceutical compositions according to this invention will be useful for regulating, e.g., for promoting or inhibiting neural growth or regeneration in the nervous system, for treating injuries or damage to nervous tissue or neurons, and for treating neural degeneration associated with injuries (such as traumas) to the nervous system, disorders or diseases, including those associated with apoptosis, necrosis or other forms of cell death.

Determination of a preferred pharmaceutical formulation and a therapeutically efficient dose regimen for a given application is within the skill of the art taking into consideration, for example, the condition and weight of the patient, the extent of desired treatment and the tolerance of the patient for the treatment. See, e.g., Handbook of Pharmaceutical Additives: An International Guide to More than 6000 Products by Trade Name, Chemical, Function, and Manufacturer, Ashgate Publishing Co., eds., M. Ash and I. Ash, 1996; The Merck Index: An Encyclopedia of Chemicals, Drugs and Biologicals, ed. S. Budavari, annual; Remington's Pharmaceutical Sciences, Mack Publishing Company, Easton, POLYAMINE; Martindale: The Complete Drug Reference, ed. K. Parfitt, 1999; and Goodman & Gilman's The Pharmaceutical Basis of Therapeutics, Pergamon Press, New York, N.Y., ed. L. S. Goodman et al.; the contents of which are incorporated herein by reference.

Administration of the neuronal PDE4 modulators of the invention, including isolated and purified forms, their salts or pharmaceutically acceptable derivatives thereof, may be accomplished using any of the conventionally accepted modes of administration of agents which are used to treat injuries or disorders, especially those relating to the central and peripheral nervous system.

Rolipram is a PDE4 inhibitor which can cross the blood-brain barrier, and thus, which can be delivered at therapeutically effective doses to an animal by subcutaneous injection. This property makes rolipram, and other PDE4 inhibitors which can cross the blood-brain barrier, a very attractive candidate as a therapeutic agent for improving neuronal growth and regeneration.

Pharmaceutical compositions comprising a PDE4 modulator of this invention may be in a variety of forms, which may be selected according to the preferred modes of administration. These include, for example, solid, semi-solid and liquid dosage forms such as tablets, capsules, pills, powders, creams, liquid solutions or suspensions, syrups, suppositories, injectable and infusible solutions, aerosols and the like. The preferred form depends on the intended mode of administration and therapeutic application. Modes of administration may include, but are not limited to, oral, parenteral (including subcutaneous, intravenous, intramuscular, intra-articular, intra-synovial, cisternal, intrathecal, intrahepatic, intralesional and intracranial injection or infusion), topical, rectal, nasal, buccal, vaginal, by inhalation, or by an implanted reservoir, external pump or catheter. In a preferred embodiment, a neuronal PDE4 modulator of the invention is administered subcutaneously, e.g., by injection. or via continuous delivery via a minipump.

The PDE4 modulatory agents of this invention may, for example, be placed into sterile, isotonic formulations with or without cofactors which stimulate uptake or stability. The formulation is preferably liquid, or may be lyophilized powder. For example, an agent of the invention may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

The compositions also will preferably include conventional 0.20 pharmaceutically acceptable carriers well known in the art (see pharmaceutical references, supra). Such pharmaceutically acceptable carriers may include other medicinal agents, carriers, including genetic carriers, adjuvants, excipients, etc., such as human serum albumin or plasma preparations. The compositions are preferably in the form of a unit dose and will usually be administered one or more times a day.

The compositions comprising a compound of this invention will contain from about 0.1 to about 90% by weight of the active compound, and more generally from about 10% to about 30%. The compositions may contain common carriers and excipients, such as corn starch or gelatin, lactose, sucrose, microcrystalline cellulose, kaolin, mannitol, dicalcium phosphate, sodium chloride and alginic acid. The compositions may contain croscarmellose sodium, microcrystalline cellulose, corn starch, sodium starch glycolate and alginic acid.

For oral administration, the pharmaceutical compositions are in the form of, for example, a tablet, capsule, suspension or liquid. Solid formulations such as tablets and capsules are particularly useful. Sustained release or enterically coated preparations may also be devised. For pediatric and geriatric applications, suspensions, syrups and chewable tablets are especially suitable. The pharmaceutical composition is preferably made in the form of a dosage unit containing a therapeutically-effective amount of the active ingredient. Examples of such dosage units are tablets and capsules.

For therapeutic purposes, the tablets and capsules which can contain, in addition to the active ingredient, conventional carriers such as binding agents, for example, acacia gum, gelatin, methylcellulose, sodium carboxymethylcellulose, polyvinylpyrrolidone (Povidone), hydroxypropyl methylcellulose, sucrose, starch and ethylcellulose sorbitol, or tragacanth; fillers, for example, calcium phosphate, glycine, lactose, maize-starch, sorbitol, or sucrose; lubricants, for example, magnesium stearate, or other metallic stearates, stearic acid, polyethylene glycol, silicone fluid, talc, waxes, oils and silica, colloidal silica or talc; disintegrants, for example, potato starch, flavoring or coloring agents, or acceptable wetting agents.

Oral liquid preparations generally are in the form of aqueous or oily solutions, suspensions, emulsions, syrups or elixirs may contain conventional additives such as suspending agents, emulsifying agents, non-aqueous agents, preservatives, coloring agents and flavoring agents. Oral liquid preparations may comprise lipopeptide micelles or monomeric forms of the lipopeptide. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For intravenous (IV) use, a water soluble form of the PDE4 modulator can be dissolved in any of the commonly used intravenous fluids and administered by infusion. Intravenous formulations may include carriers, excipients or stabilizers including, without limitation, calcium, human serum albumin, citrate, acetate, calcium chloride, carbonate, and other salts. Intravenous fluids include, without limitation, physiological saline or Ringer's solution. Polyamine and arginase modulators, optionally coupled to other carrier molecules, may also be placed in injectors, cannulae, catheters and lines.

Formulations for parenteral administration can be in the form of aqueous or non-aqueous isotonic sterile injection solutions or suspensions. These solutions or suspensions can be prepared from sterile powders or granules having one or more of the carriers mentioned for use in the formulations for oral administration. Lipopeptide micelles may be particularly desirable for parenteral administration. The compounds can be dissolved in polyethylene glycol, propylene glycol, ethanol, corn oil, benzyl alcohol, sodium chloride, and/or various buffers. For intramuscular preparations, a sterile formulation of a polyamine or arginase modulatory agent, or a suitable soluble salt form of the compound, for example a hydrochloride salt, can be dissolved and administered in a pharmaceutical diluent such as Water-for-Injection (WFI), physiological saline or 5% glucose.

Injectable depot forms may be made by forming microencapsulated matrices of the compound in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in microemulsions that are compatible with body tissues.

For topical use, the PDE4 modulatory agent of the present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient. For topical preparations, a sterile formulation of a PDE4 modulatory agent or suitable salt forms thereof, may be administered in a cream, ointment, spray or other topical dressing. Topical preparations may also be in the form of bandages that have been impregnated with a therapeutic composition.

For application to the eyes, nose or ears, the PDE4 modulatory compounds of the present invention can be presented in liquid or semi-liquid form optionally formulated in hydrophobic or hydrophilic bases as ointments, creams, lotions, paints or powders. For rectal or vaginal administration the compounds of the present invention can be administered in the form of suppositories admixed with conventional carriers such as cocoa butter, wax or other glyceride. For aerosol preparations, a sterile formulation of the peptide or lipopeptide or salt form of the compound may be used in inhalers, such as metered dose inhalers, and nebulizers.

Alternatively, the PDE4 modulatory agents of the present invention can be in powder form for reconstitution in the appropriate pharmaceutically acceptable carrier at the time of delivery. In one embodiment, the unit dosage form of the compound can be a solution of the compound or a salt thereof, in a suitable diluent in sterile, hermetically sealed ampules. The concentration of the compound in the unit dosage may vary, e.g. from about 1 percent to about 50 percent, depending on the compound used and its solubility and the dose desired by the physician. If the compositions contain dosage units, each dosage unit preferably contains from 0.1 to 10 umol/kg/hour of the active material. For adult human treatment, the dosage employed preferably ranges from 0.1 to 3.0 umol/kg/hour depending on the route and frequency of administration. For subcutaneous administration, more preferred doses are 0.15-1.5 umol/kg/hour. Doses are administered for at least 24 hours, preferably 48 hours, more preferably 3 days, more preferably 1 week, more preferably 2 weeks, more preferably 3 weeks, 1 month, 2 months or longer. Doses may be administered for periods of up to 3 months, 6 months or 12 months or longer.

The pharmaceutical compositions of this invention may also be administered using microspheres, liposomes, other microparticulate delivery systems or controlled or sustained release formulations placed in, near, or otherwise in communication with affected tissues, the bloodstream, the cerebrospinal fluid, or other locations, including muscle, which enable the targeting of the agent to an affected location in the nervous system. The compositions of the invention can be delivered using controlled (e.g., capsules) or sustained release delivery systems (e.g., bioerodable matrices). Exemplary delayed release delivery systems for drug delivery that are suitable for administration of the compositions of the invention are described in U.S. Pat. No. 4,452,775 (issued to Kent), U.S. Pat. No. 5,239,660 (issued to Leonard), U.S. Pat. No. 3,854,480 (issued to Zaffaroni).

Suitable examples of sustained release carriers include semipermeable polymer matrices in the form of shaped articles such as suppositories or microcapsules. Implantable or microcapsular sustained release matrices include polylactides (U.S. Pat. No. 3,773,319; EP 58,481), copolymers of L-glutamic acid and gamma ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

Liposomes containing PDE4 modulatory agents can be prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of agent release.

The PDE4 modulatory agents of this invention may also be attached to liposomes, which may optionally contain other agents to aid in targeting or administration of the compositions to the desired treatment site. Attachment of such agents to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

Routes of Administration

In one embodiment of the invention, cells which have been engineered to express one or more PDE4 modulatory agents of the invention may be used in therapeutic treatment regimes. Such engineered cells may be used to synthesize a therapeutic agent which can then be administered independently to a host. Alternatively, cells transformed, transfected, or infected with exogenous nucleic acid such as DNA or RNA that activates expression of a PDE4 modulatory agent of the invention that is secreted or released from the engineered cell may be used directly as a therapeutic, e.g., by implanting such engineered cells into a host at a region which is in communication with the targeted tissue or cells in need of treatment. For example, cells may be engineered to express antisense DNA, ribozymes or RNAi that specifically will target an mRNA encoding a PDE4 transcript in a nervous system cell or tissue.

Viral or non-viral gene delivery into cells which then over (or under) express a PDE4 modulatory agent according to the invention may be performed in vitro or in vivo by any of a number of techniques well known to those of skill in the art. A number of such delivery methods have been shown to work with neurons. See, e.g., Cherksey et al., U.S. Pat. No. 6,210,664 (Method for gene transfer to the central nervous system involving a recombinant retroviral expression vector); Kaplitt et al., U.S. Pat. No. 6,180,613 (AAV-mediated delivery of DNA to cells of the nervous system); Hayes et al., U.S. Pat No. 6,096,716 (Liposome-mediated transfection of central nervous system cells); Kochanek et al, U.S. Pat. No. 5,981,225 (Gene transfer vector, recombinant adenovirus particles containing same, method for producing the same and method of use of the same); Gage et al., U.S. Pat No. 5,762,926 (Method of grafting genetically modified cells to treat defects, disease or damage to the central nervous system); WO/008192 (Herpes viral vectors for gene delivery); and CA2247912 (Genetically engineered primary oligodendrocytes for transplantation-mediated gene delivery in the central nervous system); the entire disclosures of which are incorporated herein by reference.

For example, neuronal cells can be infected with a viral which causes the infected host cells to express a PDE4 modulatory agent at high levels. If the PDE4 modulatory agent is not normally a secreted protein, it can be engineered to possess a signal peptide required for secretion of a protein from a host cell. Such signal peptides are characterized by their length (about 16-30 amino acids) and hydrophobicity and which are not highly conserved at the amino acid sequence level (see, e.g., Lodish et al., Molecular Cell Biology, 3d ed., Scientific American Books, W. H. Freeman and Company, New York, 1995, Chapter 16). Amino acid residues which function as a signal sequence for secretion in a eukaryotic cell may be engineered onto the N-terminus of a heterologous protein by any of a number of routine genetic engineering methods well known to those of skill in the art. See, e.g., Farrell et al., Proteins, 41, pp.144-53 (2000) (see also http://www.healthtech.com/2001/pex); Borngraber et al., Protein Expr. Purif., 14, pp. 237-46 (1998); Collins-Racie et al., Biotechnology, 13, pp. 982-987 (1995); U.S. Pat. No. 5,747,662; WO00/50616; WO99/53059; and WO96/27016; each of which is incorporated herein by reference in its entirety. Host cells which express a secreted form of a PDE4 modulatory agent of the invention would be expected to elevate levels of cAMP in the cerebrospinal fluid (CSF) which bathes the nervous system. Alternatively, it is possible to provide a PDE4 modulatory agent, e.g., by injection, directly to the CSF. Transfected cells, secreting other forms of PDE4 modulatory agents, may be administered to a site of neuronal injury or degeneration in a similar manner.

In addition, it is possible to target endogenous genes directly by homologous recombination techniques. Such techniques allow the skilled worker to replace or modify endogenous genes in a mammalian cell—for activation, inactivation or alteration of gene coding, including intracellular targeting sequences, and non-coding (regulatory) sequences, such as transcription control sequences and other regulatory sequences which control expression levels of selected genes that modulate putrescine, polyamine or arginase activity. For homologous recombination techniques, see, e.g., U.S. Pat. Nos. 6,214,622 and 6,054,288, which are incorporated herein by reference. For polyamine regulatory sequences, see, e.g., Veress et al., Biochem. J., 346, pp. 185-191 (2000); Shantz and Pegg; Int. J. Biochem. Cell Biol., 31, pp. 107-122 (1999); Schantz et al., Cancer Res., 56, pp. 3265-3269 (1996a) and Cancer Res., 56, pp. 5136-5140 (1996b).

PDE4 modulatory agents according to the invention can also be delivered by spinal implantation (e.g., into the cerebrospinal fluid) of cells or other biocompatible materials engineered to release or secrete PDE4 modulatory agents according to this invention. Cell secretion rates or material release rates of the agent are measured in vitro (e.g., in cell culture where applicable) and then extrapolated based on relative volumes, in vivo half-lives, and other parameters understood by those of skill in the art.

Optionally, transfected cells or biocompatible delivery materials that release PDE4 modulatory agent according to the invention may be encapsulated into immunoisolatory capsules or chambers and implanted into the brain or spinal cord region using available methods that are known to those of skill in the art. See, e.g., U.S. Pat. Nos. 6,179,826, 6,083,523; 5,676,943; 5,653,975 and 5,487,739; and WO 89/04655; WO 92/19195; WO93/00127; EP 127,989; all of which are incorporated herein by reference.

Alternatively, a pump, such as one designed for subcutaneous administration, and/or a catheter-like device may be implanted at or inserted into the site of injury to administer a PDE4 modulatory agent of the invention on a timely basis and at the desired concentration, which can be selected and empirically modified by one of skill in the art. Such pharmaceutical delivery systems are well known to those of skill in the art. See, e.g., U.S. Pat. No. 4,578,057 and references cited therein; for implantable pumps, see, e.g., http://www.medtronic.com/); which are each incorporated herein by reference. As discussed above, preferably, the PDE4 modulatory agents of the invention are capable of crossing the blood brain barrier. In such cases, a pump and catheter-like device may be implanted at or inserted at a location distant from the site of injury to administer a PDE4 modulatory agent of the invention (e.g, subcutaneously) on a timely basis and at the desired concentration, which can be selected and empirically modified by one of skill in the art. In another aspect, the invention provides a pump containing the modulatory agent.

In a further aspect, this invention provides a method for treating a condition, disease or disorder associated with neuronal degeneration or lack of neuronal growth in mammals, including humans and other animals. The term “treating” is used to denote both the prevention of neuronal death and the control of axonal growth, axonal sprouting, and neural progenitor cell proliferation after the host animal has become affected. An established condition, disease or disorder may be one that is acute or chronic. The method comprises administering to the human or other animal an effective dose of a PDE4 modulatory agent of the invention. An effective dose of rolipram, for example, is generally between about 0.1 to 10 umol/kg/hour of rolipram, or rolipram-related analogs or derivatives, or pharmaceutically acceptable salts thereof. For an adult human patient of approximately 70 kg, this would give a dose of 7.0 to 700 umol of rolipram/hour, which would be 168 to 16,800 umol dose per day. In a preferred embodiment, the effective dose of a PDE4 inhibitor, particularly a PDE4 specific inhibitor, is one that inhibits PDE4 activity by at least 40%, more preferably 50%, 60%, 70%, 80%, 90% or 95% in a neuron or nervous system tissue or organ that is being treated.

The PDE4 modulatory agent of the invention may be administered alone or as part of a combination therapy. A preferred dose is from about 0.1 to 10 umol/kg/hour (2.4 to 240 umol/kg/day) of rolipram, rolipram-related analogs or derivatives, or pharmaceutically acceptable salts thereof. A more preferred dose is from about 0.1 to 3.0 umol/kg/hour (2.4 to 48 umol/kg/day) rolipram, rolipram-related analogs or derivatives, or pharmaceutically acceptable salts thereof. These dosages for rolipram may be used as a starting point by one of skill in the art to determine and optimize effective dosages of other PDE4 inhibitors and of the invention.

In one embodiment, the invention provides a method for treating a condition, disease or disorder associated with neuronal degeneration or lack of neuronal growth in a subject with a therapeutically-effective amount of a PDE4 modulator of the invention. Exemplary procedures for delivering agents to the nervous system are described, e.g., in Cherskey et al., U.S. Pat. No. 6,210,664; Kaplitt et al., U.S. Pat. No. 6,180,613; Hayes et al., U.S. Pat. No. 6,096,716; Kochanek et al, U.S. Pat. No. 5,981,225; Gage et al., U.S. Pat. No. 5,762,926; and CA2247912; the entire contents of which are incorporated herein by reference in their entirety.

As used herein the phrase “therapeutically-effective amount” means an amount of a PDE4 modulator of the invention, such that the subject shows a detectable improvement in neuronal growth or regeneration after being treated under the selected administration regime (e.g., the selected dosage levels and times of treatment). The term “treating” is defined as administering, to a subject, a therapeutically-effective amount of a compound of the invention, to prevent the occurrence of or to control or eliminate symptoms associated with a condition, disease or disorder associated with neuronal death or lack of neuronal growth. The term “subject”, as described herein, is defined as a mammal or a cell in culture. In a preferred embodiment, a subject is a human or other animal patient in need of treatment.

A compound of the invention can be administered alone, or in combination with other compounds (e.g., a “cocktail”), including but not limited to other compounds of the invention. A compound of the invention may be administered as a single daily dose or in multiple doses per day. Preferably, the treatment regime will include administration of a PDE4 modulator over extended periods of time, e.g., for several days or for from two to four weeks. The amount per administered dose or the total amount administered will depend on such factors as the nature and severity of the symptoms, the age and general health of the patient, the tolerance of the patient to the treatment program, factors which may be determined empirically.

Phosphodiesterases

Although cAMP was the first intracellular second messenger identified (Sutherland, 1970), our understanding of the complex system of enzymes that generate, regulate, detect and break down cAMP is far from complete.

Mammalian cells can synthesize up to nine isoforms of adenylyl cyclase, the enzyme which synthesizes cAMP. In the mammalian cell, cAMP is degraded (hydrolyzed) by a family of enzymes called phosphodiesterases (PDE). There are many isoforms of PDE, including isoform 4 (“PDE4” or Type 4). See, e.g., Takahashi et al., J. Neurosci., Jan. 15, 1999;19(2):610-8; Duplantier et al., J. Med. Chem. Jan. 5, 1996;39(1):120-5. The PDEs constitute a diverse group of enzymes. The level of complexity of PDEs matches and probably even surpasses that of adenylyl cyclases because PDEs provide the cells an additional opportunity for crosstalk between the different cAMP dependent signaling pathways.

The first cAMP phosphodiesterase gene was identified in the fruit fly, Drosophila, in a screen for genes which affect memory deficiency. (Dudai et al., 1976). In 1981, it was demonstrated biochemically that the gene, named “dunce”, carried a mutation in cAMP phosphodiesterase (Byers et al., 1981). The Drosophila dunce gene was cloned (Davis and Davidson, 1986) and subsequently, mammalian homologs of the dunce gene were cloned and characterized (Davis et al., 1989). They later were shown to be the members of the PDE4 family of enzymes.

Phosphodiesterases—Type 4 (PDE4)

The PDE 4 family of enzymes consists of four enzymes (PDE A-D), three of which (PDE4A, PDE4B and PDE4D) are expressed in the nervous system (Perez-Torres et al., 2000). All enzymes of the PDE4 family are cAMP specific and they are inhibited by rolipram. The pattern of transcription and splicing of PDE4 changes with development (Davies et al., 1989). Two features are really exceptional. The first is the extent of similarity to the Drosophila cDNAs (75% identity), which indicates that the PDE4 genes are highly conserved genes. The second is the complexity of the rat PDE4 genes. The PDE 4A gene, for example, is 49 kb long and has 16 exons. Each gene can encode up to six splice variants.

All PDE4 proteins have a similar basic structure, containing a conserved catalytic domain at the COOH terminus, and a choice of two upstream conserved regions at the amino terminus of the protein (Bolger et al., 1997). Combination of these upstream conserved regions, as well as the extreme amino terminus regions which are unique to each protein, targets these enzymes to their intended cell destination, and further, confers on these PDE4 enzymes their distinctive regulatory properties. One of the most evident differences in these splice variants is their subcellular distribution. Long isoforms, that possess both upstream conserved regions, ucr1 and ucr2, are associated with the membranes and the short forms are usually cytosolic. The nervous system expresses mostly the long isoforms of these enzymes (Bolger et al., 1997).

Molecular cloning of PDE4 genes was a starting point for the cloning of other families of PDEs. As of today, the list of mammalian phosphodiesterases has 19 genes subdivided into 10 different PDE families. (see, e.g., Soderling et al. (2000), Curr Opin Cell Biol. 12:174-9, herein incorporated by reference). Almost all of these PDEs are expressed at various levels in the nervous system. Activity of PDE4, however, is responsible for at least 70% of the total cAMP PDE activity in the brain (Jin et al., 1999). Experiments with inhibitors of PDE in different tissues have demonstrated that only in neurons are cAMP levels elevated significantly after applying PDE4-specific inhibitors. In other tissues, a combination of inhibitors of different PDE families is required (Shirotani et al., 1991). This supports the notion that, in other tissues, the relative contribution of PDE4 is not as high as in the nervous system.

Rolipram

Rolipram is a specific inhibitor of PDE4. Rolipram has been the subject of clinical trials as an antidepressant, an anti-inflammatory, a memory improving agent and as a sedative. In studies of rolipram as a memory-improving agent, very low concentrations of the drug were used (0.1-3.0 umol/kg) (Barad et al., 1998). When injected subcutaneously at a dose of 0.1 umol/kg, rolipram improved the performance of mice in a hippocampus-dependent memory task. At concentrations of up to 0.3 umol/kg, rolipram did not raise basal cAMP levels in hippocampal slices in vitro, Increased basal cAMP levels could be detected at doses of 1.0-3.0 umol/kg. Interestingly, higher doses of rolipram, 3.0 umol/kg, which caused an increase in basal cAMP levels, did not have memory improving effects. No side effects of rolipram were reported at these concentrations (Barad et al., 1998).

Rolipram was also reported to have anti-inflammatory and sedative effects at higher concentrations. Sedative effects of rolipram were demonstrated in rats at concentrations of 5-10 umol/kg (Silvestre et al., 1999). Studies of rolipram as an anti-inflammatory drug in a rat model of arthritis used rolipram at 20 umol/kg (Francischi et al., 2000; Hogan et al., 2001). No side effects were reported at these higher concentrations. Rolipram's anti-inflammatory effect has also been demonstrated in an animal model for multiple sclerosis, which is an autoimmune inflammatory disease (Genain et al., Proc. Natl. Acad. Sci., 92: 3601-3605 (1995)).

All references cited herein are hereby incorporated by reference.

In order that this invention may be more fully understood, the following examples are set forth to illustrate methods of this invention used to identify the PDE modulatory agents which inhibit myelin and MAG's developmentally regulated effect on neurite growth, compositions of this invention which comprise such agents, and methods comprising the administration of those compositions. These examples are for the purpose of illustration only and are not to be construed as limiting the scope of the invention in any way.

### EXAMPLES

The following examples show that elevating cAMP levels in mammalian neurons using a PDE4 specific inhibitor, rolipram, enables adult axons to grow in the presence of myelin inhibitors. The experiments show that a) rolipram added directly to the media of cultured cerebellar neurons improves neurite outgrowth of those neurons in the presence of inhibitory MAG; b) priming with rolipram enables cerebellar neurons to grow in the presence of inhibitory MAG and myelin; c) rolipram injected or delivered by minipumps subcutaneously to an animal blocks inhibition of axonal outgrowth by MAG of isolated cerebellar (CNS) neurons from postnatal day 12 (P12) and 14 (P14) rats and of DRG neurons (PNS) from P30 rats, with the blockage of inhibition increasing over time; and d) rolipram delivered by minipumps subcutaneously to an animal promotes motor neuron recovery in vivo after spinal cord transection.

### Example 1

### Direct Treatment of Cerebellar Neurons with dbcAMP or Rolipram

The response of neurons to the inhibitors of axonal outgrowth (e.g., MAG, myelin) is dependent on the intracellular levels of cAMP. We thus wanted to see whether addition of rolipram, a specific inhibitor of PDE4, would enable neurons to grow in the presence of MAG.

Cerebellar neurons from P5 rats were isolated as described previously (Cai et al., 1999) Briefly, cerebellum was treated with 0.025% of trypsin, triturated and incubated for 10 min at 37° C. Trypsinization was stopped by adding an equal amount of DMEM containing 10% fetal calf serum (FCS). Cells were centrifuged at 800 rpm for 5 min. The cells were resuspended to a single-cell suspension in 2 ml of SATO (see Cai et al., 1999, herein incorporated by reference). The concentration of cells were adjusted to 6×104 cells/ml. Cells were plated in SATO media onto a monolayer of either MAG-expressing Chinese hamster ovary (CHO) cells or onto a monolayer of control CHO cells (i.e., which do not express MAG) and cultured (see also U.S. Pat. No. 5,932,542). Where indicated, dbcAMP (1 mM) or rolipram (0.1 uM, 0.25 uM, 0.5 uM or 1.0 uM) was added to the media. After 18 hours of culture, neurons were fixed and immunostained with a rabbit polyclonal antibody against glial acidic protein 43 (GAP43) to visualize the neurites. The length of the longest neurite of the first two hundred GAP43-positive neurons was determined using the Simple 32™ software (see Cai et al., 1999). The mean length of a neurite was determined and presented as the average length+/−SEM in micrometers (um). Results are expressed as a percentage of neurite length from neurons grown in the absence of dbcAMP or rolipram.

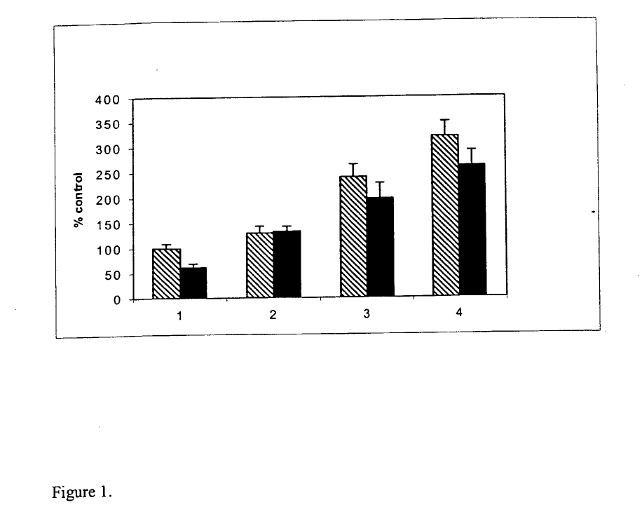

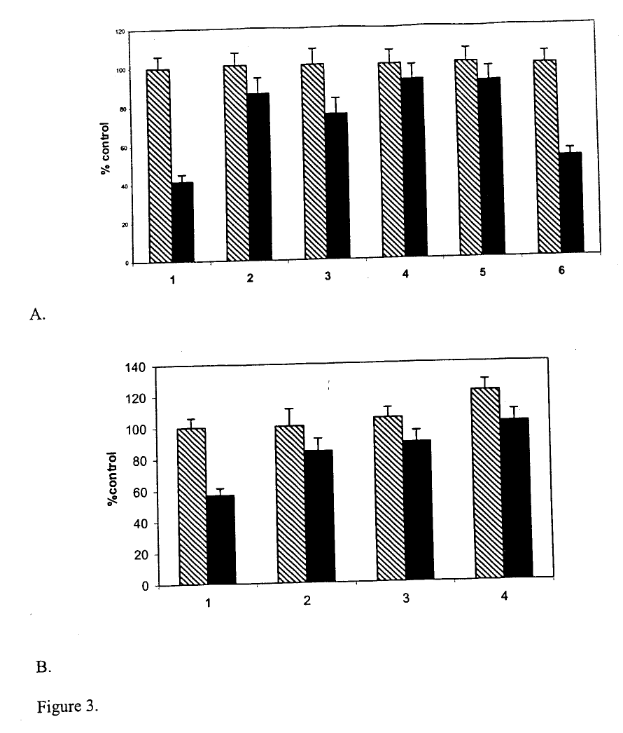

As shown in FIG. 1, rolipram in the range of concentrations 0.25 uM-1.0 uM, partially blocks the inhibition of axonal outgrowth by MAG. At a concentration of 0.5 uM, rolipram blocked the inhibition of axonal outgrowth by MAG with an efficiency of 80% compared to dibutyryl-cAMP (db-cAMP) (FIG. 3). The effect of rolipram is dose-dependent.

### Example 2

### Priming of Cerebellar Neurons with BDNF or Rolipram

We had previously shown that treating neurons with the neurotrophins brain-derived neurotrophic factor (BDNF) and nerve growth factor (NGF) for 6-18 hours prior to the encounter with inhibitors of axonal outgrowth (MAG and myelin), termed “priming”, conferred upon the neurons the ability to grow in the presence of MAG and myelin in vitro (Cai et al., 1999) and to regenerate in vivo (Bregman, 1998). The levels of cAMP in the neurons were elevated after priming with neurotrophins. Thus, we sought to determine whether priming with rolipram would be as effective as priming with BDNF in blocking myelin and MAG-mediated inhibition of axonal outgrowth.

Isolated cerebellar neurons in SATO media (prepared as in Example 1) were plated onto poly-L-lysine-coated dishes at 1×10⁶ cells/dish. Where indicated, either BDNF at a concentration of 200 ng/ml, or rolipram at a concentration 0.1 uM or 0.25 uM (all from Sigma) was added. After culture for 18 hours (termed priming), neurons were removed with 0.1% trypsin. Trypsinization was stopped by adding 5 ml of DMEM containing 10% FCS. The primed neurons were centrifuged at 800 rpm for 6 min and resuspended in SATO media. The concentration of cells were adjusted to 6×10⁴ cells/ml. Neurons were plated immediately onto either MAG-expressing CHO cells, onto control CHO cells (which do not express MAG), or onto purified, immobilized myelin. Myelin was prepared as described previously (Cai et al., 1999) from rat CNS white matter. Neurons were cultured overnight before being fixed and immunostained for GAP43 to visualize the neurites. The length of the longest neurite per neuron, from 180-200 neurons, was measured. Results are presented as the average length+/−SEM in micrometers (um). (See, e.g., U.S. Pat. Nos. 5,932,542 and 6,203,792).

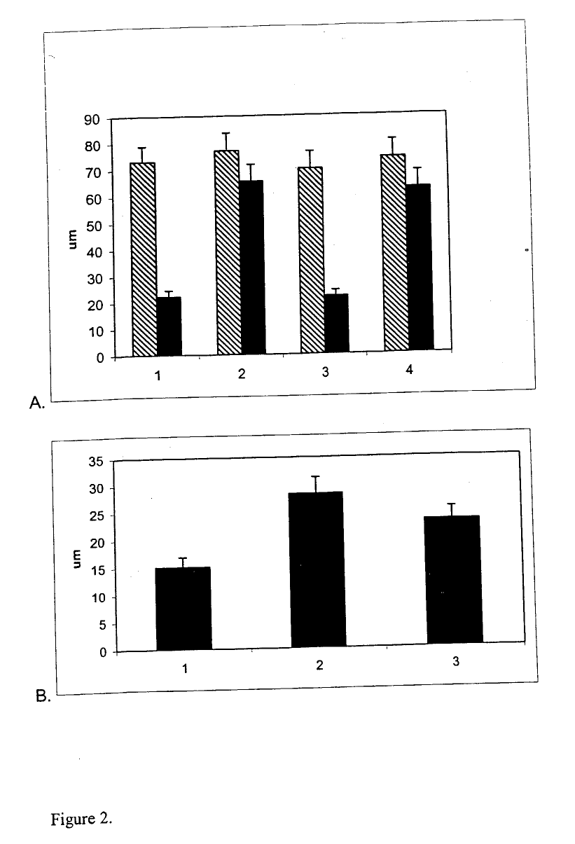

As shown in FIG. 2, priming of cerebellar neurons with rolipram at a concentration of 0.25 uM was almost as effective as priming cerebellar neurons with BDNF in blocking inhibition of axonal outgrowth by MAG. The length of neurites on the monolayer of CHO cells expressing MAG after treatment with rolipram was 95% of the neuronal length of neurons treated with BDNF). These results demonstrate that rolipram, a PDE4 specific inhibitor, is an agent which can reverse MAG and myelin-mediated inhibition of neural growth in cultured neurons in a dose-dependent manner.

### Example 3

### Subcutaneous Delivery of Rolipram to Rats by Injection or Minipumps; Effects on Neuronal Growth and Regeneration

A. Subcutaneous Rolipram Injections

In order to determine whether a PDE4 specific inhibitor could be used to prime neurons in vivo and prevent inhibition of neurite outgrowth by myelin and MAG, rolipram was injected subcutaneously into P12 and P30 rats every 3 hours for 24 hours. For all experiments, rolipram was dissolved in DMSO and sterile saline was added to adjust the concentrations. The final volume of the rolipram solution was 0.2 ml for each injection. At each time point, a single rolipram injection was given. Rolipram was injected subcutaneously with insulin syringes (Becton Dickinson 1 ccU-100 insulin Syringe) under the skin of the rat's neck (for all experiments) and in various other regions (only for first experiment using P12 rats). Control animals were injected with a 0.2 ml mixture of DMSO and sterile saline without rolipram, following the same schedule.

Rolipram was injected subcutaneously into P12 rats at concentrations of 0, 7.5, 20, 25 or 40 nmol/kg every 3 hours for 24 hours before sacrificing. Cerebellar neuron and DRG neurons were isolated from control and treated animals and plated onto a monolayer of MAG-expressing CHO cells or a monolayer of control CHO cells which do not express MAG. Cerebellar neurons were isolated as described in Example 1. Dorsal root ganglia (DRG) neurons were isolated as described previously (De Bellard et al., 1996). Briefly, ganglia were removed from the animals and incubated in 5 ml of SATO media containing 0.025% of trypsin and 0.15% of collagenase type I (Worthington) for 1 hour at 37° C. The ganglia were triturated and trypsinization was stopped by adding 5 ml of DMEM containing 10% FCS. Ganglia were centrifuged at 800 rpm for 6 min, and resuspended in SATO. Neurons were cultured overnight on CHO monolayers as described in Example 1 before being fixed and immunostained for GAP43 to visualize the neurites, as described in Example 1. As a positive control, some neurons from control animals were also cultured overnight in the presence of 1 mM dbcAMP. The length of the longest neurite per neuron, from 180-200 neurons, was measured and results are the average length+/−SEM. Results are expressed as a percentage of growth of the neurons isolated from control animals, treated with saline/DMSO injections and plated on control CHO cells, without dbcAMP or rolipram treatment. See FIGS. 3A and 3B.

The results of this experiment show that, at a dose of 7.5 nmol/kg, rolipram effectively blocks subsequent inhibition of neuronal growth by MAG. At a dose of 25 nmol/kg, inhibition of neuronal growth by MAG is essentially completely blocked. Importantly, these results demonstrate that subcutaneous injection of the PDE4 inhibitor rolipram into an animal can raise the endogenous levels of cAMP in neurons in vivo to a level sufficient to overcome the normal growth inhibition by MAG. We saw similar results when isolated neurons were cultured on purified myelin rather than MAG as a neural growth inhibitor. Further, injections of rolipram for 24 hours did not affect neurite length for control cells. See, e.g., FIGS. 3A and 3B.

These results demonstrate for the first time that inhibition of the enzyme PDE4 with the specific inhibitor, rolipram, can overcome inhibition of mammalian axonal outgrowth by MAG and myelin. Importantly, the results in FIGS. 3A and 3B show that, when administered subcutaneously to live animals, rolipram has similar effects on two different populations of neurons—cerebellar neurons in the CNS and DRG neurons in the PNS. To have this effect, rolipram must have reached these neurons and crossed the blood brain barrier. The growth state of mature neurons can thus be altered—and inhibition of neuronal growth and regeneration overcome in vivo after spinal cord or other CNS (or PNS) injury—by subcutaneous injections with a PDE4 inhibitor that crosses the blood brain barrier, such as rolipram.

In a second series of experiments, we injected increasing concentrations of rolipram subcutaneously into older animals (postnatal day 30; P30) every 3 hours for 24 hours and, as above, studied neurite outgrowth of isolated DRG neurons in the presence or absence of MAG.

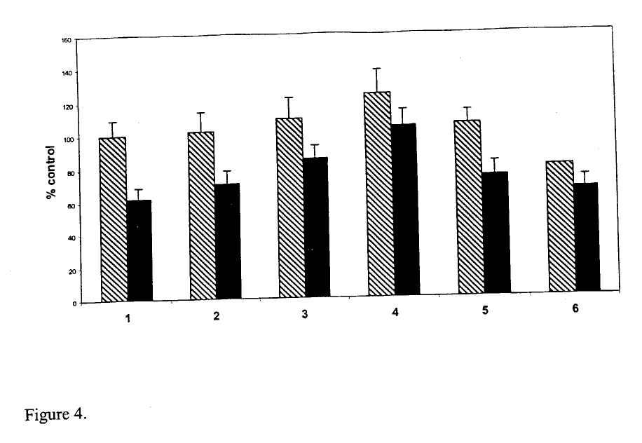

DRG neurons were isolated from control and treated P30 animals and plated onto a monolayer of MAG-expressing CHO cells or a monolayer of control CHO cells. Neurons were cultured overnight before being fixed and immunostained for GAP43 to visualize the neurites, as described in Example 1. The length of the longest neurite per neuron from 180-200 neurons was measured. Results are expressed as a percentage of growth of the neurons isolated from control animals and are shown as the average length+/−SEM presented as a percentage of growth on control cells. See FIG. 4.

In older animals, the inhibition of axonal outgrowth by MAG was also blocked (i.e., relieved) by rolipram in a dose-dependent manner (FIG. 4). The dose of rolipram that was most effective when injected subcutaneously in P30 rats was 0.5 umol/kg, significantly higher than the most effective subcutaneous dose of rolipram we observed for the P12 rats (20 nmol/kg). Thus, the most effective dose of rolipram (by subcutaneous injection) for relieving myelin or MAG neuronal growth inhibition appears to depend both on the age and the weight of the animal subject.

We next determined whether the effects of rolipram were time dependent. Rolipram (0.5 umol/kg) was administered by repeated subcutaneous injections to P30 rats for increasing amounts of time. DRG neurons were isolated from control and treated animals and plated onto a monolayer of MAG-expressing CHO cells or a monolayer of control CHO cells. Neurons were cultured overnight before being fixed and immunostained for GAP43 to visualize the neurites. The lengths of the longest neurite per neuron from 180-200 neurons were measured. Results are expressed as a percentage of growth of the neurons isolated from control animals and are shown as the average length+/−SEM presented as percentage of growth on control cells.

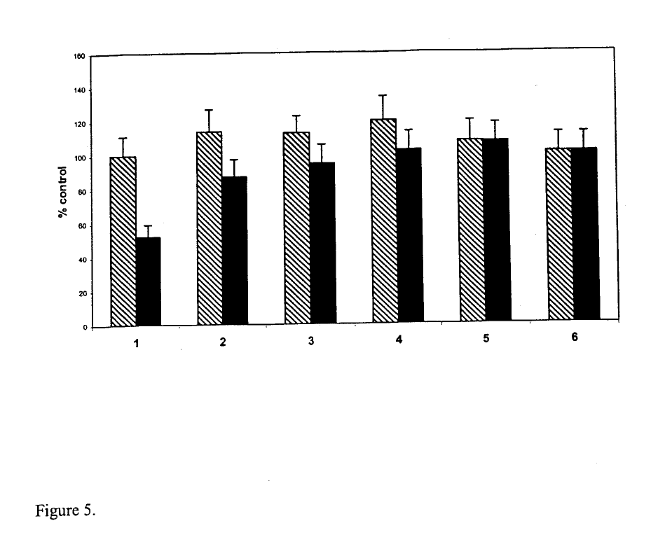

FIG. 5 shows the results of a time course of the effects of repeated subcutaneous rolipram injections (0.5 umol/kg) on the ability of DRG neurons isolated from treated P30 rats to grow in the absence or presence of MAG. The effects of rolipram were time-dependent. The length of the axons of the DRG neurons isolated from the P30 animals treated with rolipram for 24 hours was increased in comparison to the length of the neurons treated for 6 hours. However, there appeared to be no significant difference between the effects of 1, 2 and 3 days of treatment with rolipram, when administered by intermittent subcutaneous injections, on the length of the neurites.

B. Continuous Subcutaneous Rolipram Delivery by Minipumps

We repeated the time course experiments discussed above using minipumps to deliver subcutaneous rolipram continuously to P30 animals. Continuous delivery of rolipram by mini-pumps provides a stable concentration of the drug in the body of the subject.

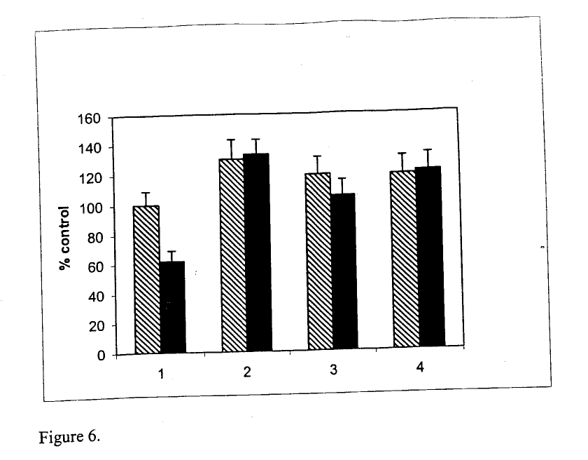

Rolipram was delivered subcutaneously with ALZET 2001 minipumps. Minipumps were inserted subcutaneously under the skin of the animals' backs. Two minipumps were used for each animal; the combined flow rate was 2 ul/hour. Rolipram was dissolved in DMSO and sterile saline was added to adjust the doses of rolipram that were released from minipumps every hour. After 24 hours of treatment, DRG neurons were isolated from control and treated animals and plated onto a monolayer of MAG-expressing CHO cells or a monolayer of control CHO cells. Neurons were cultured overnight before being fixed and immunostained for GAP43 to visualize the neurites. The length of the longest neurite per neuron from 180-200 neurons was measured. Results are expressed as a percentage of growth of the neurons isolated from control animals and are shown as the average length+/−SEM presented as percentage of growth on control cells. See FIG. 6.

After 24 hours of treatment, DRG neurons isolated from continuously treated animals were no longer inhibited by MAG. We found that a continuous dose of 0.4 umol/kg/hour rolipram delivered subcutaneously to P30 rats was optimal for subsequent neuronal growth and regeneration.

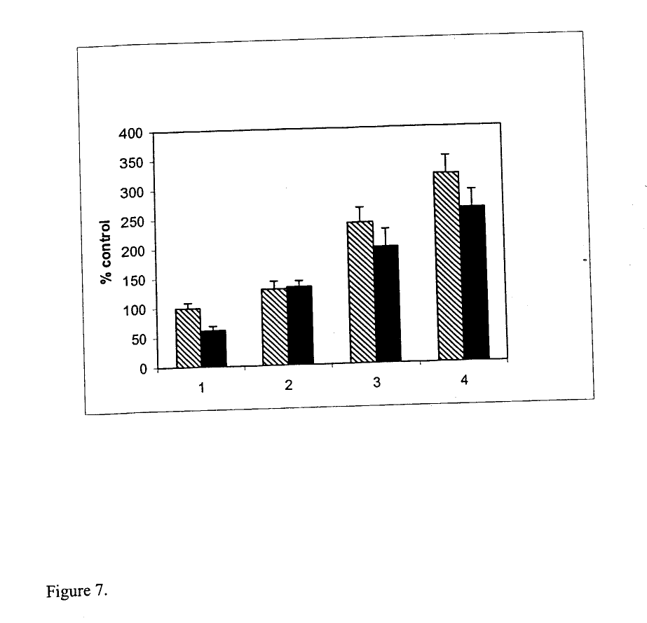

In order to determine whether longer continuous treatment with a PDE4 inhibitor would increase the relief of inhibition of neurite outgrowth by myelin or MAG, rolipram was administered subcutaneously by minipump (0.4 umol/kg/hour). After 1, 2 or 3 days of continuous rolipram treatment, DRG neurons were isolated from control and treated animals and plated onto a monolayer of MAG-expressing CHO cells or a monolayer of control CHO cells. Neurons were cultured overnight before being fixed and immunostained for GAP43 to visualize the neurites. The length of the longest neurite per neuron from 180-200 neurons was measured. Results are expressed as a percentage of growth of the neurons isolated from control animals and are shown as the average length+/−SEM, presented as a percentage of growth on control cells. See FIG. 7.

After 24 hours of treatment, DRG neurons isolated from continuously treated animals were no longer inhibited by MAG. After 2 days of continuous rolipram treatment, axonal outgrowth was significantly promoted both in the presence of MAG and on control CHO cells. Axonal outgrowth was even further promoted after 3 days of continuous rolipram treatment. This suggests that continuous administration of a PDE4 specific inhibitor for a prolonged period of time not only overcomes the inhibition of MAG and myelin on neurite outgrowth but, surprisingly, is also highly effective for promoting neurite outgrowth compared to neurons in the absence of a PDE4 specific inhibitor.

### Example 4

### Continuous Subcutaneous Delivery of Rolipram to Rats After Spinal Cord Injury; Effects on Motor Recovery

One strategy being pursued for promoting axonal regeneration after spinal cord injury is implantation of Schwann cells into sites of spinal cord injury to support axonal growth. (See, e.g., Xu et al., 1999; Ramon-Cueto et al., 1998; Guest, J. D. et al., 1997; Xu et al., 1997). The adult rat spinal cord is either subjected to a moderate contusive injury or a complete transection and Schwann cell grafts are introduced into the site of injury. Neurotrophic factor in combination with Schwann cell grafts have recently been shown to improve axonal extension after injury. (Jones L. L. et al., 2001; Menei P. et al., 1998). We used this model system to study the effects of continuous rolipram delivery on the ability of a rat with a spinal cord injury to recover motor function.

A. Complete Transection of the Spinal Cord

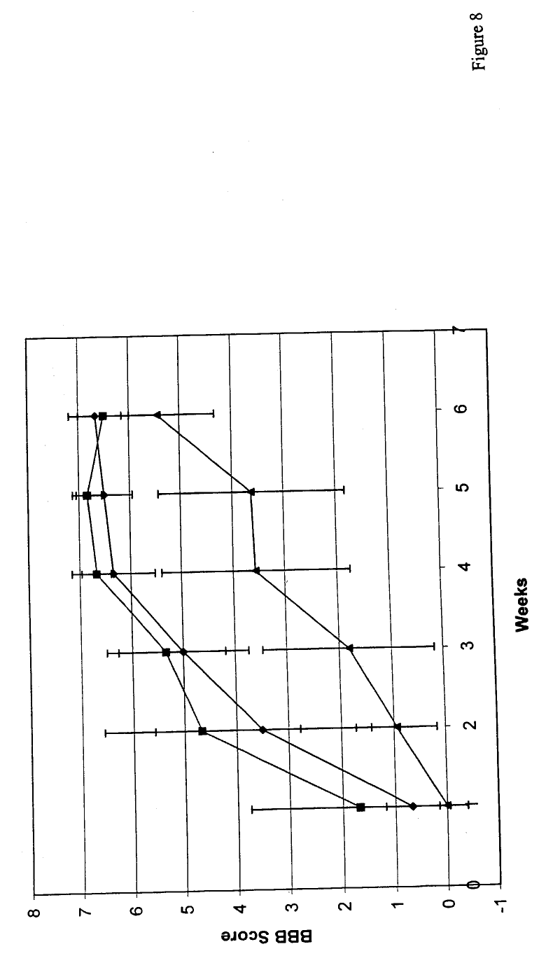

Adult rat spinal cords were completely transected at the T8 cord level and the next caudal segment was removed (Xu et al. (1997), herein incorporated by reference). Schwann cells were purified in culture from adult rat sciatic nerve, suspended in Matrigel: DMEM (30:70), and drawn into 8 mm long polymeric guidance channels at a density of 120×10⁶ cells/ml. Xu et al. (1997). Each cut stump was inserted 1 mm into the channel. At the time of transection, a Schwann cell bridge was implanted at the injury site and rolipram was delivered subcutaneously via minipump at 0.07 umol/kg/hour for two weeks as described in Example 3B. As a negative control, animals were delivered saline only. As a positive control, 5 ul of 10 mM dbcAMP was infused in the proximal and distal stump of the lesion in animals. Animals were assessed on a weekly basis for hindlimb locomotion, which is a measure of their motor recovery, using the BBB test. See FIG. 8.

The results shown in FIG. 8 demonstrate that administration of rolipram significantly increases the BBB score (i.e., motor recovery) for animals having a complete transection of the spinal cord, and further show that rolipram has essentially the same result as administration of dbcAMP.

B. Moderate Contusive Injury to the Spinal Cord

Adult rat spinal cord were exposed and injured with a weight drop device (NYU). See Beattie et al. (1997) and Basso et al. (1996). At the same time as the injury was inflicted, rolipram was delivered subcutaneously via minipumps at 0.07 nmol/kg/hour for 2 weeks. One week after injury, Schwann cells, which had been grown and purified in culture, were injected into the lesion site along with 4 injections, each of 0.2 ul, of 1 mM dbcAMP. As a negative control, animals were injected with Schwann cells alone. Other animals were administered dbcAMP and Schwann cells. One group of animals was administered four injections, each of 0.2 ul, of 1 mM dbcAMP one week after injury. Another group was administered four injections, each of 0.2 ul, of 50 mM dbcAMP one week after injury. Another group was administered four injections, each of 0.2 ul, of 50 mM dbcAMP one day after injury. Animals were assessed on a weekly basis for hindlimb locomotion using the BBB test.

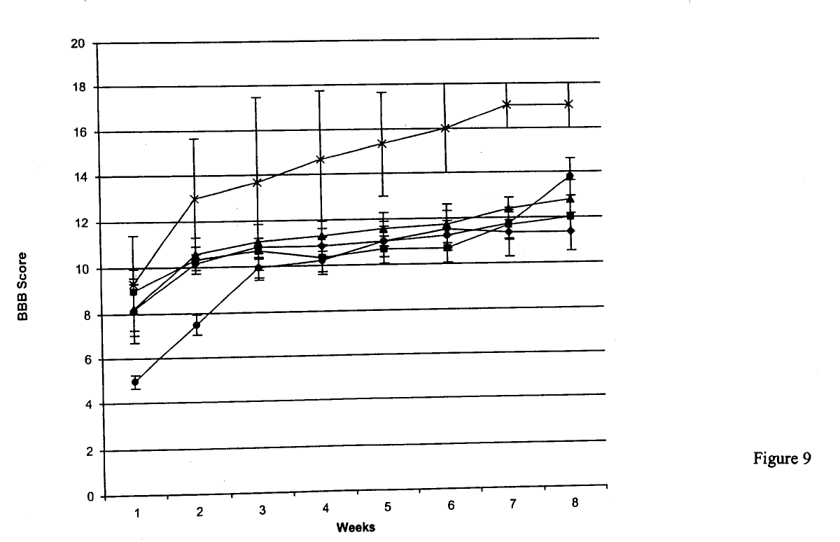

As shown in FIG. 9, compared to the corresponding controls, continuous rolipram delivery before and after Schwann cell implants significantly improved motor skill recovery in spinal cord injured rats even as early as 2 weeks after injury, with effects improving up to 7 weeks after injury, as measured by the BBB score. Significantly, a BBB score of greater than 15 is scored as complete recovery, a score which was only achieved in the group of rats that received continuous rolipram treatment after spinal cord injury.

### Example 5

### Continuous Subcutaneous Delivery of Rolipram to Rats Reduces the Formation of a Glial Scar After Spinal Cord Injury

The spinal cord of P30 rats is completely transected as described in Example 4A. At the time of transection, rolipram is delivered continuously for 1, 2, 3, 4, 5, 6, and 7 days as described in Example 3. Control animals are administered saline only. After a further 3 weeks, the animals are sacrificed and the spinal cord removed. The section surrounding the lesion site, consisting of 10-20 mm proximal and distal, is fixed, sectioned and immunostained for glial fibrillary acidic protein (GFAP) or for chondroitin sulphate proteoglycans (CSPS). In addition, in a separate group of rolipram-treated and control animals, a similar section of spinal cord, surrounding the lesion site, is fixed for electron microscopy. Immunostaining for GFAP and CSPG, and morphology at the EM level are compared in the rolipram-treated and the control animals.

### REFERENCES

The following references are incorporated herein by reference.

Allen R. A., Merriman M. W., Perry M. J., and Owens R. J. 1999. Development of a recombinant cell-based system for the characterisation of phosphodiesterase 4 isoforms and evaluation of inhibitors. Biochem Pharmacol. 57:1375-82.

Bach M E, Barad M, Son H, Zhuo M, Lu Y F, Shih R, Mansuy I, Hawkins R D, Kandel E R. Free in Age-related defects in spatial memory are correlated with defects in the late phase of hippocampal long-term potentiation in vitro and are attenuated by drugs that enhance the cAMP signaling pathway. Proc Natl Acad Sci U S A. Apr. 27, 1999;96(9):5280-5.

Baillie, G. S., S. J. MacKenzie, I. McPhee, and M. D. Houslay. 2000. Subfamily selective actions in the ability of Erk2 MAP kinase to phosphorylate and regulate the activity of PDE4 cyclic AMP-specific phosphodiesterases. Br J Pharmacol. 131:8119.

Barad, M., R. Bourtchouladze, D. G. Winder, H. Golan, and E. Kandel. 1998. Rolipram, a type IV-specific phosphodiesterase inhibitor, facilitates the establishment of long-lasting long-term potentiation and improves memory. Proc Natl Acad Sci U S A. 95:15020-5.

Basso D. M., Beattie M. S., and Bresnahan J. C. 1996. Graded histological and locomotor outcomes after spinal cord contusion using the NYU weight-drop device versus transection. Exp Neurol. Jun;139:244-56.

Bates, C. A., and D. J. Stelzner. (1993). Extension and regeneration of corticospinal axons after early spinal injury and the maintenance of corticospinal topography. Exp Neurol. 123:106-17.

Beattie M. S., Bresnahan J. C., Komon J., Tovar C. A., Van Meter M., Anderson D. K., Faden A. I., Hsu C. Y., Noble L. J., Salzman S., and Young W. 1997. Endogenous repair after spinal cord contusion injuries in the rat. Exp Neurol. 148:453-63.

Bolger, G. B., S. Erdogan, R. E. Jones, K. Loughney, G. Scotland, R. Hoffmann, I. Wilkinson, C. Farrell, and M. D. Houslay. (1997). Characterization of five different proteins produced by alternatively spliced mRNAs from the human cAMP-specific phosphodiesterase PDE4D gene. Biochem J. 328:539-48.

Bracken, M. B., Aldrich, E. F., Herr, D. L., Hitchon, P. W., Holford, T. R., Marshall, L. F., Nockels, R. P., Pascale, V., Shepard, M. J., Sonntag, V. K., et al. (2000). Clinical measurement, statistical analysis, and risk-benefit: controversies from trials of spinal injury, J Trauma 48, 558-61.

Bregman, B. S. (1987). Development of serotonin immunoreactivity in the rat spinal cord and its plasticity after neonatal spinal cord lesions. Brain Res. 431:245-63.

Bregman, B. S., Kunkel-Bagden, E., Schnell, L., Dai, H. N., Gao, D., and Schwab, M. E. (1995). Recovery from spinal cord injury mediated by antibodies to neurite growth inhibitors, Nature 378, 498-501.

Bregman, B. S. (1998). Regeneration in the spinal cord. Curr Opin Neurobiol. 8:800-7.

Byers, D., R. L. Davis, and J. A. Kiger, Jr. (1981). Defect in cyclic AMP phosphodiesterase due to the dunce mutation of learning in Drosophila melanogaster. Nature. 289:79-81.

Cai, D., Y. Shen, M. De Bellard, S. Tang, and M. T. Filbin. (1999). Prior exposure to neurotrophins blocks inhibition of axonal regeneration by MAG and myelin via a cAMP-dependent mechanism. Neuron. 22:89-101.

Cai, D., J. Qui, Z. Cao, M. McAtee, B. Bregman and M. T. Filbin. (2001). Neuronal Cyclic AMP Controls the Developmental Loss in Ability of Axons to Regenerate. J. Neurosci. 21(13):4731-4739.

Chen, M. S., A. B. Huber, M. E. van der Haar, M. Frank, L. Schnell, A. A. Spillmann, F. Christ, and M. E. Schwab. 2000. Nogo-A is a myelin-associated neurite outgrowth inhibitor and an antigen for monoclonal antibody IN-1 [see comments]. Nature. 403:434-9.

David, S., and A. J. Aguayo. 1981. Axonal elongation into peripheral nervous system “bridges” after central nervous system injury in adult rats. Science. 214:931-3.

Davis, R. L., and N. Davidson. 1986. The memory gene dunce+encodes a remarkable set of RNAs with internal heterogeneity. Mol Cell Biol. 6:1464-70.

Davis, R. L., H. Takayasu, M. Eberwine, and J. Myres. 1989. Cloning and characterization of mammalian homologs of the Drosophila dunce+gene. Proc Natl Acad Sci U S A. 86:3604-8.

DeBellard, M. E., S. Tang, G. Mukhopadhyay, Y. J. Shen, and M. T. Filbin. 1996. Myelin-associated glycoprotein inhibits axonal regeneration from a variety of neurons via interaction with a sialoglycoprotein. Mol Cell Neurosci. 7:89-101.

Dudai, Y., Y. N. Jan, D. Byers, W. G. Quinn, and S. Benzer. 1976. dunce, a mutant of Drosophila deficient in learning. Proc Natl Acad Sci U S A. 73:1684-8.

Dugan L., Kim S J., Zhang Y., Bart R., Sun Y., Holtzman D., Gutman D. Differential effects of cAMP in neurons and astrocytes. Role of B-Raf. The Journal of Biological Chemistry, 1999, v.274, No36, pp.25842-25848

Francischi, J. N., C. M. Yokoro, S. Poole, W. L. Tafuri, F. Q. Cunha, and M. M. Teixeira. 2000. Anti-inflammatory and analgesic effects of the phosphodiesterase 4 inhibitor rolipram in a rat model of arthritis. Eur J Pharmacol. 399:243-9.

Grewal, S. S., R. D. York, and P. J. Stork. 1999. Extracellular-signal-regulated kinase signalling in neurons. Curr Opin Neurobiol. 9:544-53.

Hasan, S. J., H. S. Keirstead, G. D. Muir, and J. D. Steeves. 1993. Axonal regeneration contributes to repair of injured brainstem-spinal neurons in embryonic chick. J Neurosci. 13:492-507.

Hermanns S, Reiprich P, Muller H W. A reliable method to reduce collagen scar formation in the lesioned rat spinal cord. J Neurosci Methods Sep. 30, 2001;110(1-2):141-6.

Hoffmann, R., G. S. Baillie, S. J. MacKenzie, S. J. Yarwood, and M. D. Houslay. 1999. The MAP kinase ERK2 inhibits the cyclic AMP-specific phosphodiesterase HSPDE4D3 by phosphorylating it at Ser579. EMBO J. 18:893-903.

Hoffmann, R., I. R. Wilkinson, J. F. McCallum, P. Engels, and M. D. Houslay. 1998. cAMP-specific phosphodiesterase HSPDE4D3 mutants which mimic activation and changes in rolipram inhibition triggered by protein kinase A phosphorylation of Ser-54: generation of a molecular model. Biochem J. 333:139-49.

Hogan, C. J., G. D. Fang, W. M. Scheld, J. Linden, and D. R. Diduch. 2001. Inhibiting the inflammatory response in joint sepsis. Arthroscopy. 17:311-315.

Howland, D. R., B. S. Bregman, A. Tessler, and M. E. Goldberger. 1995. Transplants enhance locomotion in neonatal kittens whose spinal cords are transected: a behavioral and anatomical study. Exp Neurol. 135:123-45.

Huang, D. W., McKerracher, L., Braun, P. E., and David, S. (1999). A therapeutic vaccine approach to stimulate axon regeneration in the adult mammalian spinal cord, Neuron 24, 639-47.

Jin, S. L., F. J. Richard, W. P. Kuo, A. J. D'Ercole, and M. Conti. 1999. Impaired growth and fertility of cAMP-specific phosphodiesterase PDE4D-deficient mice. Proc Natl Acad Sci U S A. 96:11998-2003.

Jones, L. L., M. Oudega, M. B. Bunge and M. H. Tuszynski. 2001. Neurotrophic factors, cellular bridges and gene therapy for spinal cord injury. J. Physiol. 533:83-89.

Lehmann, M., Fournier, A., Selles-Navarro, I., Dergham, P., Sebok, A., Leclerc, N., Tigyi, G., and McKerracher, L. (1999). Inactivation of Rho signaling pathway promotes CNS axon regeneration, J Neurosci 19, 7537-47.

Li, Y., and G. Raisman. 1993. Long axon growth from embryonic neurons transplanted into myelinated tracts of the adult rat spinal cord. Brain Res. 629:115-27.

Ma L, Zablow L, Kandel E R, Siegelbaum S A. Cyclic AMP induces functional presynaptic boutons in hippocampal CA3-CA1 neuronal cultures. Nat Neurosci. Jan. 2, 1999;2(1):24-30.

MacKenzie, S. J., G. S. Baillie, I. McPhee, G. B. Bolger, and M. D. Houslay. 2000. ERK2 mitogen-activated protein kinase binding, phosphorylation, and regulation of the PDE4D cAMP-specific phosphodiesterases. The involvement of COOH-terminal docking sites and NH2-terminal UCR regions. J Biol Chem. 275:16609-17.

McKeon, R. J., A. Hoke, and J. Silver. 1995. Injury-induced proteoglycans inhibit the potential for laminin-mediated axon growth on astrocytic scars. Exp Neurol. 136:32-43.

McKerracher, L., S. David, D. L. Jackson, V. Kottis, R. J. Dunn, and P. E. Braun. 1994. Identification of myelin-associated glycoprotein as a major myelin-derived inhibitor of neurite growth. Neuron. 13:805-11.

McKerracher, L. (2001). Spinal cord repair: strategies to promote axon regeneration, Neurobiol. Dis 8, 11-8.

Menei P., C. Montero-Menei, S. R. Whittemore, R. P Bunge and M. B. Bunge. 1998. Sxhwann cells genetically modified to secrete human BDNF promote enhanced axonal regrowth across transected adult rat spinal cord. Eur. J. Neurosci. 10(2):607-21.

Mukhopadhyay, G., P. Doherty, F. S. Walsh, P. R. Crocker, and M. T. Filbin. 1994. A novel role for myelin-associated glycoprotein as an inhibitor of axonal regeneration. Neuron. 13:757-67.

Neumann, S., and C. J. Woolf. 1999. Regeneration of dorsal column fibers into and beyond the lesion site following adult spinal cord injury [see comments]. Neuron. 23:83-91.

Perez-Torres, S., X. Miro, J. M. Palacios, R. Cortes, P. Puigdomenech, and G. Mengod. 2000. Phosphodiesterase type 4 isozymes expression in human brain examined by in situ hybridization histochemistry and[3H]rolipram binding autoradiography. Comparison with monkey and rat brain. J Chem Neuroanat. 20:349-74.

Ramon-Cueto, A., G. W. Plant, J. Avila and M. B. Bunge. 1998. Long-distance axonal regeneration in the transected adult rat spinal cord is promoted by olfactory ensheathing glia transplants. J. Neurosci. 18(10):3803-15.

Richardson, P. M., U. M. McGuinness, and A. J. Aguayo. 1980. Axons from CNS neurons regenerate into PNS grafts. Nature. 284:264-5.

Schnell, L., and Schwab, M. E. (1990). Axonal regeneration in the rat spinal cord produced by an antibody against myelin-associated neurite growth inhibitors, Nature 343, 269-72.

Seidl, E. C. (1999). Promising pharmacological agents in the management of acute spinal cord injury, Crit Care Nurs Q 22, 44-50.

Sette C., Conti M. 1996, Phosphorylation and activation of a cAMP-specific phosphodiesterase by the cAMP-dependant protein kinase. The Journal of Biological chemistry 271 (28): 16526-16534.

Shewan, D., M. Berry, and J. Cohen. 1995. Extensive regeneration in vitro by early embryonic neurons on immature and adult CNS tissue. J Neurosci. 15:2057-62.

Shirotani M., Yui Y, Hattori R., Kawai C. 1991,U-61,431-F, a stable prostacyclin analoque, inhibits prolifiration of bovine ascular smooth muscle cells with little antiproliferative effect on endothelial cells. Prostaglandines 41:97-110.

Silvestre, J. S., A. G. Fernandez, and J. M. Palacios. 1999. Preliminary evidence for an involvement of the cholinergic system in the sedative effects of rolipram in rats. Pharmacol Biochem Behav. 64:1-5.

Spillmann, A. A., C. E. Bandtlow, F. Lottspeich, F. Keller, and M. E. Schwab. 1998. Identification and characterization of a bovine neurite growth inhibitor (bNI-220). J Biol Chem. 273:19283-93.

Sutherland, E. W. 1970. On the biological role of cyclic AMP. Jama. 214:1281-8.

Xu X. M., Chen A., Guenard V., Kleitman N., Bunge M. B. 1997. Bridging Schwann cell transplants promote axonal regeneration from both the rostral and caudal stumps of transected adult rat spinal cord. J Neurocytol. 26:1-16.

Yao, H., R. D. York, A. Misra-Press, D. W. Carr, and P. J. Stork. 1998. The cyclic adenosine monophosphate-dependent protein kinase (PKA) is required for the sustained activation of mitogen-activated kinases and gene expression by nerve growth factor. J Biol Chem. 273:8240-7.

Yoon, D. H., Kim, Y. S., and Young, W. (1999). Therapeutic time window for methylprednisolone in spinal cord injured rat, Yonsei Med J 40, 313-20.

York, R. D., H. Yao, T. Dillon, C. L. Ellig, S. P. Eckert, E. W. McCleskey, and P. J. Stork. 1998. Rap1 mediates sustained MAP kinase activation induced by nerve growth factor. Nature. 392:622-6.

Zu X. M., A. Chen, V. Guenart, N. Kleitman and M. B. Bunge. 1997. Bridging Schwann cell transplants promote axonal regeneration from both the rostral and caaudal stumps of transected adult rat spinal cord. J. Neurocytol., 26(1):1-16.

Zu, X. M., S. X. Zhang, H. Li, P. Aebischer and M. B. Bunge. 1999. Regrowth of axons in the distal spinal cord through a Schwann-cell-seeded mini-channel implanted into hemisected adult rat spinal cord. Eur. J. Neurosci. 11(5): 1723-40.

We claim:

1. A composition comprising an amount of a phosphodiesterase type 4 (PDE4) inhibitor effective to inhibit phosphodiesterase type 4 activity in a neuron when administered subcutaneously to a mammal for a prolonged period of time.

2. A composition comprising an amount of a PDE4 inhibitor effective to increase cAMP levels in a neuron when administered subcutaneously to a mammal for a prolonged period of time.

3. A composition comprising an amount of a PDE4 inhibitor which, when administered subcutaneously to a mammal for a prolonged period of time, promotes neuronal growth in the presence of MAG or myelin.

4. A composition according to any one of claims 1-3, wherein the PDE4 inhibitor is rolipram.

5. A composition according to any one of claims 1-3, wherein the PDE4 inhibitor is administered at a dose of 0.1 to 10 umol/kg/hour, wherein said dose is administered for at least 24 hours.

6. The composition according to claim 5, wherein the PDE4 inhibitor is administered at a dose of 0.1 to 3 umol/kg/hour.

7. The composition according to claim 5, wherein the PDE4 inhibitor is administered for at least a period of time selected from the group consisting of 48 hours, 72 hours, 96 hours, one week, two weeks, one month, two months, three months, six months and twelve months.

8. A method for regulating neural growth or regeneration in the nervous system of a mammal, comprising the step of administering to said mammal for a prolonged period of time a composition comprising a therapeutically effective amount of an agent that inhibits PDE4 activity in a neuron of the mammal.

9. A method for the treatment of an injury or damage to nervous tissue or neurons in a patient in need thereof, comprising the step of administering to said patient for a prolonged period of time a composition comprising a therapeutically effective amount of an agent that inhibits PDE4 activity in a neuron.

10. A method for treating neural degeneration associated with disorders or diseases in a patient in need thereof, comprising the step of administering to the patient for a prolonged period of time a composition comprising a therapeutically effective amount of an agent which inhibits PDE4 activity in a neuron.

11. A method for treating a disease, disorder or condition associated with apoptosis in a patient in need thereof comprising the step of administering to the patient for a prolonged period of time a composition comprising a therapeutically effective amount of an agent which inhibits PDE4 activity in a neuron.

12. A method for treating a neurodegenerative disease selected from the group consisting of amyotrophic lateral sclerosis, Alzheimer's disease, Parkinson's disease, and Huntington's disease, in a patient in need thereof, said method comprising the step of administering for a prolonged period of time to said patient a composition comprising a therapeutically effective amount of an agent which inhibits PDE4 activity in a neuron.

13. The method of any one of claims 9-12, wherein the PDE4 inhibitor is administered continuously or repeatedly for at least 24 hours.

14. The method of claim 13, wherein the PDE4 inhibitor is administered continuously or repeatedly for at least a period of time selected from the group consisting of 48 hours, 72 hours, 96 hours, one week, two weeks, one month, two months, three months, six months and twelve months.

15. The method of any one of claims 8-14, wherein the PDE4 inhibitor is administered subcutaneously.

16. The method of any one of claims 8-15, wherein the PDE4 inhibitor is rolipram.

17. The method of any one of claims 8-16, wherein the patient is a human subject.

18. The method according to any one of claims 8-17, wherein the PDE4 inhibitor is administered at a dose of 0.1 to 10 umol/kg/hour.

19. The method according to claim 18, wherein the PDE4 inhibitor is administered at a dose of 0.1 to 3 umol/kg/hour.
